SEQUENCE LISTING (4 sequences)

SEQ ID NO: 1 - protein, 12 aa, Artificial Sequence
  C-TAT
CYGRKKRRQRRR

SEQ ID NO: 2 - protein, 12 aa, Artificial Sequence
  TAT-C
YGRKKRRQRRRC

SEQ ID NO: 3 - DNA, 16 bp, Artificial Sequence
  AntiSurvivin LNA
ctcaatccatggcagc

SEQ ID NO: 4 - protein, 11 aa, Artificial Sequence
  TAT
YGRKKRRQRRR

(19) United States
(12) Patent Application Publication
(10) Pub. No.: US 2010/0203066 A1
(43) Pub. Date: Aug. 12, 2010

(54) POLYMERIC LINKERS CONTAINING PYRIDYL DISULFIDE MOIETIES

(75) Inventors: Hong Zhao, Edison, NJ (US); Prasanna Reddy, Somerset, NJ (US); Jing Xia, Warren, NJ (US)

Correspondence Address: LUCAS & MERCANTI, LLP, 475 PARK AVENUE SOUTH, 15TH FLOOR, NEW YORK, NY, 10016, US

(73) Assignee: ENZON PHARMACEUTICALS, INC., Bridgewater, NJ (US)

(21) Appl. No.: 12/674,006

(22) PCT Filed: Sep. 15, 2007
(86) PCT No.: PCT/US07/78596

§ 371 Date: Mar. 8, 2010

Related U.S. Application Data
(60) Provisional application No. 60/956,814, filed on Aug. 20, 2007.

(51) Int. Cl. A61K 39/44; C07D 213/71; A61K 31/44; C07K 16/18; C07H 21/00; A61K 31/713; C07K 5/08; A61K 38/06; C07K 14/00; A61K 38/16; C07K 7/00; A61K 38/04; C07K 2/00; A61P 35/00; C12N 9/96

(52) U.S. Cl. 424/180.1; 546/294; 514/345; 530/391.9; 536/23.1; 514/44 R; 530/331; 514/18; 530/350; 514/12; 530/327; 514/15; 530/300; 435/188

(57) ABSTRACT

The present invention provides polymeric linkers containing pyridyl disulfide moieties. Methods of making the polymeric linkers and methods of making conjugates using the same are also disclosed.

### CROSS-REFERENCE TO RELATED APPLICATION

This application claims the benefit of priority from U.S. Provisional Patent Application Ser. No. 60/956,814 filed Aug. 20, 2007, the contents of which are incorporated herein by reference.

### FIELD OF THE INVENTION

The present invention relates to drug delivery systems. In particular, the invention relates to activated polymer-based drug delivery linkers containing pyridyl disulfide moiety which improve conjugation of thiol containing biologically active moieties.

### BACKGROUND OF THE INVENTION

Over the years, numerous methods have been proposed for delivering therapeutic agents into the body and improving bioavailability of those medicinal agents. One of the attempts is to include such medicinal agents as part of a soluble transport system. Such transport systems can include permanent conjugate-based systems or prodrugs. In particular, polymeric transport systems can improve the solubility and stability of medicinal agents. For example, the conjugation of water-soluble polyalkylene oxides with therapeutic moieties such as proteins and polypeptides is known. See, for example, U.S. Pat. No. 4,179,337 (the '337 patent), the disclosure of which is incorporated herein by reference. The '337 patent discloses that physiologically active polypeptides modified with PEG circulate for extended periods in vivo, and have reduced immunogenicity and antigenicity.

Additional improvements have been also realized. For example, polymer-based drug delivery platform systems containing benzyl elimination systems, trialkyl lock systems, etc. were disclosed by Enzon Pharmaceuticals as a means of releasably delivering proteins, peptides and small molecules. See also Greenwald, et al., J. Med. Chem. Vol. 42, No. 18, 3657-3667; Greenwald, et al., J. Med. Chem. Vol. 47, No. 3, 726-734; Greenwald, et al., J. Med. Chem. Vol. 43, No. 3, 475-487. The contents of each of the foregoing are hereby incorporated herein by reference.

More recently, polyethylene glycol (PEG) has been proposed for conjugation with a wide variety of biologically active compounds including oligonucleotides, targeting proteins, peptides, etc. For the conjugation, the hydroxyl end-groups of the polymer must first be converted into reactive functional groups. This process is frequently referred to as “activation” and the product is called an “activated polyalkylene oxide”. Other polymers are similarly activated. There are several functional groups known in the art for this purpose.

In spite of the attempts and advances, further improvements in PEG and polymer conjugation technology for thiol containing moieties have therefore been sought. The present invention addresses this need and others.

### SUMMARY OF THE INVENTION

In order to overcome the above problems and improve the technology for drug delivery, there are provided new branched polymers and conjugates made therewith.

In one aspect of the invention, there are provided compounds of Formula (I):

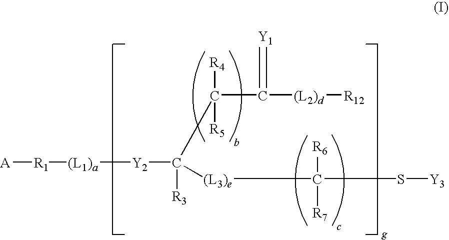

wherein:

R₁ is a substantially non-antigenic water-soluble polymer;

A is a capping group or

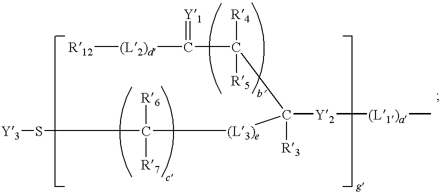

Y₁ and Y′₁ are independently S, O, or NR₂;

Y₂ and Y′₂ are independently S, O, SO, SO₂, NR₂₀;

Y₃ and Y′₃ are independently H, leaving group, activating group, functional group, or

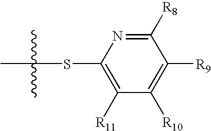

L₁₋₃ and L′₁₋₃ are independently selected bifunctional linkers;

R₂₋₁₁, R′₂₋₁₁, and R₂₀ are independently selected from among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆heteroalkyl, C₁₋₆alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;

R₁₂ and R′₁₂ are independently selected from among hydrogen, hydroxyl, leaving group, functional group, medicinal agent, targeting agent, diagnostic agent, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆heteroalkyl, C₁₋₆alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted arylcarbonyloxy, maleimidyl, vinyl, substituted sulfone, amino, carboxy, mercapto, hydrazide and carbazate;

(a), (a′), (d) and (d′) are independently zero or a positive integer, preferably zero or 1;

(b) and (b′) are independently zero or a positive integer, preferably zero or an integer from 1 to 10, more preferably zero or 1, and most preferably 0;

(c) and (c′) are independently zero or a positive integer, preferably zero or an integer from 1 to 10, more preferably zero or 1, and most preferably 1;

(e) and (e′) are independently zero or 1;

(g) and (g′) are independently zero or 1, preferably 1;

provided that (a) and (g) are not simultaneously zero.

In certain preferred aspects of the invention, the polymeric drug-delivery systems include cysteine.

In some preferred aspects, at least one of R₈₋₁₁ or R′₈₋₁₁ is an electron-withdrawing group such as substituted amido, acyl, azido, carboxy, alkyloxycarbonyl, cyano, and nitro, preferably nitro, and more preferably nitro group as R₈ or R′₈.

In another preferred aspects, R₁₂ or R′₁₂ is selected from among medicinal agent, targeting agent, or diagnostic agent.

In some particularly preferred aspects, R₁ includes a linear or branched poly(ethylene glycol) residue with molecular weight of from about 5,000 to about 60,000, Y₁ and Y′₁ are O, Y₂ and Y′₂ are NR₂₀, (a) and (a′) are zero or 1, (b) and (b′) are zero or 1, (c) and (c′) are 1, and (e) and (e′) are zero. In one particular aspect, R₂₋₇, R′₃₋₇, R₉₋₁₁ and R′₉₋₁₁ are selected from among hydrogen, methyl and ethyl, and each is more preferably hydrogen.

In another aspect of the invention, there are provided methods of preparing the compounds described herein, methods of using the compound of invention further for conjugation with a biologically active compound, and methods of using the resulting conjugates for treatment.

One advantage of the pyridyl disulfide moiety containing polymeric transport systems described herein is that the artisans are able to conjugate thiol containing moiety selectively. Even incorporating an amino acid having a thiol as part of the polymeric activated system, the compounds of the current invention can also provide a starting point for the peptide synthesis. A further advantage of the polymeric systems described herein allows attaching a second agent. Multiple substitutions can be introduced by utilizing a branching moiety as the linker providing the disulfide bond. The multiple substitution of the compound of the invention will further provide the artisans in the art to be able to attach a second drug to have synergistic effect for therapy on top of a targeting group which can selectively conjugate via disulfide bond. The polymeric delivery systems described herein allow targeting medicinal agents into the site of treatment.

For purposes of the present invention, the terms “a biologically active moiety” and “a residue of a biologically active moiety” shall be understood to mean that portion of a biologically active compound which remains after the biologically active compound has undergone a substitution reaction in which the transport carrier portion has been attached.

Unless otherwise defined, for purposes of the present invention:

the term “alkyl” shall be understood to include straight, branched, substituted, e.g. halo-, alkoxy-, and nitro-C₁₋₁₂ alkyls, C₃₋₈ cycloalkyls or substituted cycloalkyls, etc.;

the term “substituted” shall be understood to include adding or replacing one or more atoms contained within a functional group or compound with one or more different atoms;

the term “substituted alkyls” include carboxyalkyls, aminoalkyls, dialkylaminos, hydroxyalkyls and mercaptoalkyls;

the term “substituted cycloalkyls” include moieties such as 4-chlorocyclohexyl; aryls include moieties such as napthyl; substituted aryls include moieties such as 3-bromophenyl; aralkyls include moieties such as toluoyl; heteroalkyls include moieties such as ethylthiophene;

the term “substituted heteroalkyls” include moieties such as 3-methoxy-thiophene; alkoxy includes moieties such as methoxy; and phenoxy includes moieties such as 3-nitrophenoxy;

the term “halo” shall be understood to include fluoro, chloro, iodo and bromo; and

the terms “sufficient amounts” and “effective amounts” for purposes of the present invention shall mean an amount which achieves a therapeutic effect as such effect is understood by those of ordinary skill in the art.

### BRIEF DESCRIPTION OF THE DRAWINGS

FIG. 1 schematically illustrates methods of synthesis described in Examples 1-5.

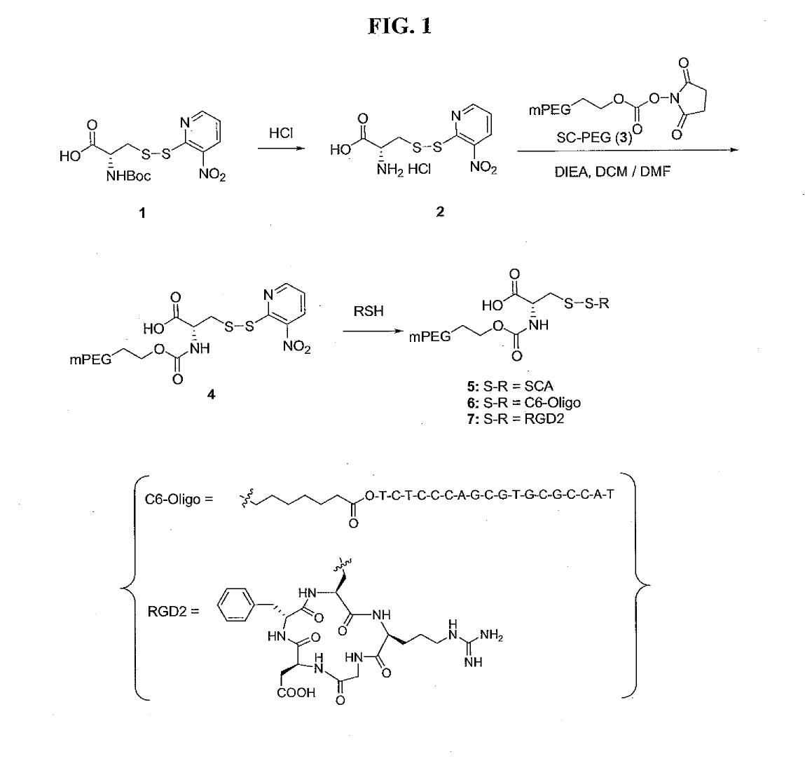

FIG. 2 schematically illustrates methods of synthesis described in Examples 6-8.

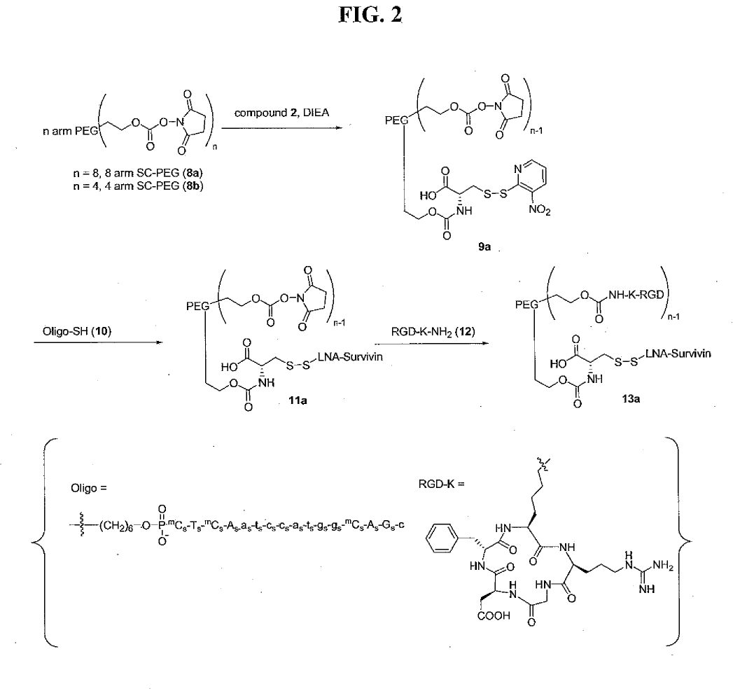

FIG. 3 schematically illustrates methods of synthesis described in Examples 7-12.

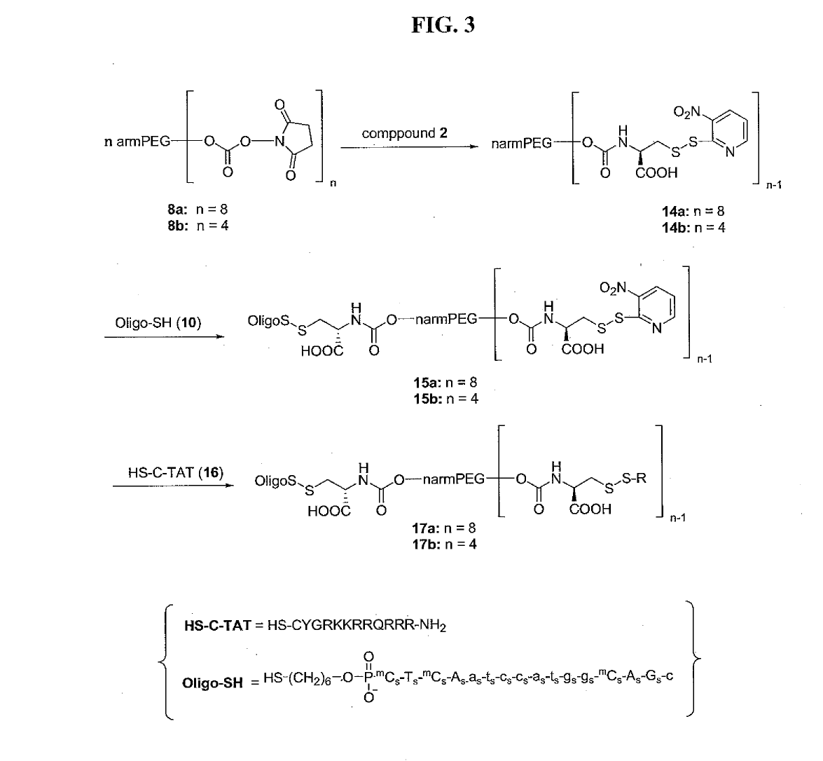

FIG. 4 schematically illustrates methods of synthesis described in Examples 13-15.

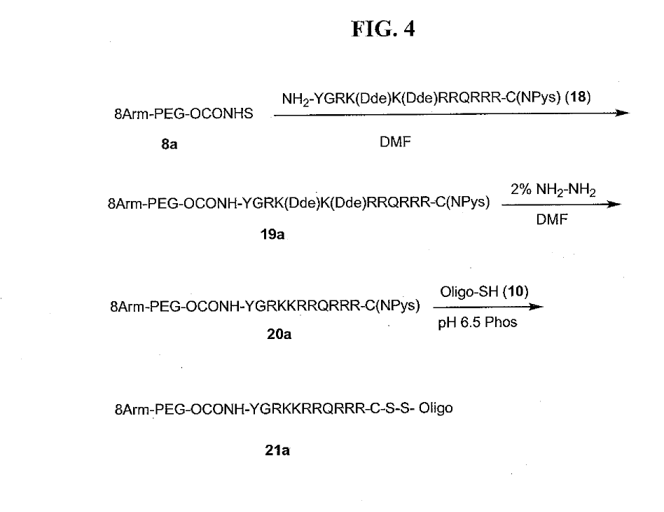

### DETAILED DESCRIPTION OF THE INVENTION

### A. Overview

In one aspect of the present invention, there are provided compounds of Formula (I):

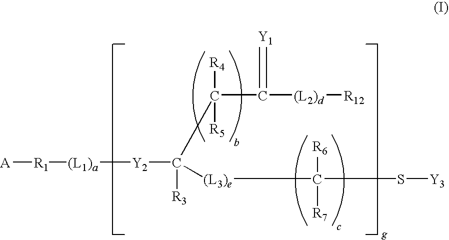

wherein:

R₁ is a substantially non-antigenic water-soluble polymer;

A is a capping group or

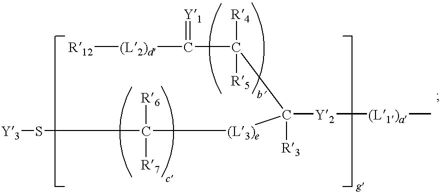

Y₁ and Y′₁ are independently S, O, or NR₂;

Y₂ and Y′₂ are independently S, O, SO, SO₂, NR₂₀;

Y₃ and Y′₃ are independently H, leaving group, activating group, functional group, or

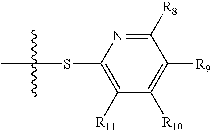

L₁₋₃ and L′₁₋₃ are independently selected bifunctional linkers;

R₂₋₁₁, R′₂₋₁₁, and R₂₀ are independently selected from among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;

R₁₂ and R′₁₂ are independently selected from among hydrogen, hydroxyl, leaving group, functional group, medicinal agent, targeting agent, diagnostic agent, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆heteroalkyl, substituted C₁₋₆heteroalkyl, C₁₋₆ alkoxy, aryloxy, C₁₋₆heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted arylcarbonyloxy, maleimidyl, vinyl, substituted sulfone, amino, carboxy, mercapto, hydrazide and carbazate;

(a), (a′), (d) and (d′) are independently zero or a positive integer, preferably zero or 1;

(b) and (b′) are independently zero or a positive integer, preferably zero or an integer from 1 to 10, more preferably zero or 1, and most preferably 0;

(c) and (c′) are independently zero or a positive integer, preferably zero or an integer from 1 to 10, more preferably zero or 1, and most preferably 1;

(e) and (e′) are independently zero or 1;

(g) and (g′) are independently zero or 1, preferably 1;

provided that (a) and (g) are not simultaneously zero.

Within those aspects of the invention, the substituents contemplated for substitution, where the moieties corresponding to R₂₋₁₁, R′₂₋₁₁, and R₂₀ are indicated as being possibly substituted can include, for example, acyl, amino, amido, amidine, ara-alkyl, aryl, azido, alkylmercapto, arylmercapto, carbonyl, carboxylate, cyano, ester, ether, formyl, halogen, heteroaryl, heterocycloalkyl, hydroxy, imino, nitro, thiocarbonyl, thioester, thioacetate, thioformate, alkoxy, phosphoryl, phosphonate, phosphinate, silyl, sulfhydryl, sulfate, sulfonate, sulfamoyl, sulfonamide, and sulfonyl.

In one aspect of the invention, the leaving group is selected from among OH, halogens, activated esters, cyclic imide thione, N-hydroxysuccinimidyl, para-nitrophenoxy, N-hydroxyphtalimide, N-hydroxybenzotriazolyl, imidazole, tosyl, mesyl, tresyl, nosyl, C₁₋₆ alkyloxy, C₁₋₆ alkanoyloxy, arylcarbonyloxy, ortho-nitrophenoxy, para-nitrophenoxy, pentafluorophenoxy, 1,3,5-trichlorophenoxy and 1,3,5-trifluorophenoxy.

In another aspect of the invention, the biological moieties include —NH₂ containing moieties, —OH containing moieties and —SH containing moieties.

In yet another aspect, A can be selected from among H, NH₂, OH, CO₂H, C₁₋₆ alkoxy, and C₁₋₆ alkyls. In some other preferred embodiments, A can be methyl, ethyl, methoxy, ethoxy, H, and OH. A is more preferably methyl or methoxy.

In certain preferred aspects of the invention, the polymeric drug-delivery systems include cyteine or other thiol containing amino acids.

In some preferred aspects, at least one of R₈₋₁₁ or R′₈₋₁₁ is an electron-withdrawing group such as substituted amido, acyl, azido, carboxy, alkyloxycarbonyl, cyano, and nitro, preferably nitro, and more preferably nitro group as R₈ or R′₈.

In another preferred aspects, R₁₂ or R′₁₂ is selected from among medicinal agent, targeting agent, or diagnostic agent.

In some particularly preferred aspects, R₁ includes a linear or branched poly(ethylene glycol) residue with molecular weight of from about 5,000 to about 60,000, Y₁ and Y′₁ are O, Y₂ and Y′₂ are NR₂₀, (a) and (a′) are zero or 1, (b) and (b′) are zero or 1, (c) and (c′) are 1, and (e) and (e′) are zero. In one particular aspect, R₂₋₇, R′₃₋₇, R₉₋₁₁ and R′₉₋₁₁ are selected from among hydrogen, methyl and ethyl, and each is more preferably hydrogen.

In one preferred embodiment, compounds described herein have the formula

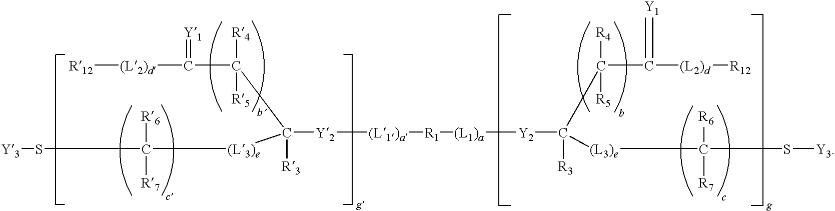

In some preferred embodiments, compounds described herein have the formula (II)

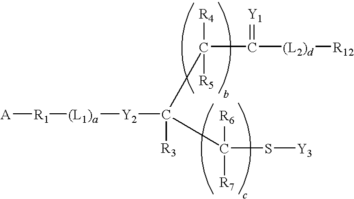

wherein

A₁ is a capping group or

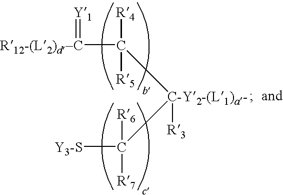

all other variables are the same as defined above.

In one preferred embodiment, compounds described herein have the formula

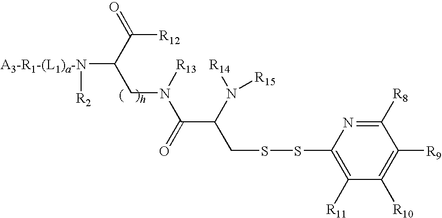

wherein:

A₃ is a capping group or

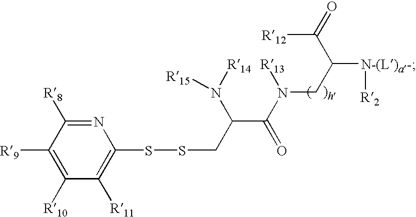

(h) and (h′) are independently zero or a positive integer, preferably zero to 10, and more preferably zero to 4; and

all other variables are the same as defined above.

In more preferred embodiments, compounds described herein can be, for example,

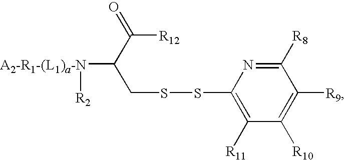

wherein,

A₂ is a capping group or

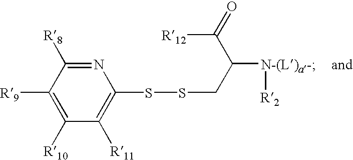

all other valuables are as previously defined.

In some preferred embodiments, R₂₋₁₁, R′₂₋₁₁, and R₂₀ are independently hydrogen or CH₃. In some particularly preferred embodiments, R₂₋₁₁, R′₂₋₁₁, and R₂₀ are all hydrogen. In yet other particular embodiments, Y₁₋₂ and Y′₁₋₂ include O and NR₂₀, and R₂₋₁₁, R′₂₋₁₁, and R₂₀ includes hydrogen, C₁₋₆ alkyls, cycloalkyls, aryls, and aralkyl groups.

### B. Substantially Non-Antigenic Water-Soluble Polymers

Polymers employed in the compounds described herein are preferably water soluble polymers and substantially non-antigenic such as polyalkylene oxides (PAO's).

In one aspect of the invention, the compounds described herein include a linear, terminally branched or multi-armed polyalkylene oxide. In some preferred embodiments of the invention, the polyalkylene oxide includes polyethylene glycol and polypropylene glycol.

The polyalkylene oxide has an average molecular weight from about 2,000 to about 100,000 daltons, preferably from about 5,000 to about 60,000 daltons. The polyalkylene oxide can be more preferably from about 5,000 to about 25,000 or alternatively from about 20,000 to about 45,000 daltons. In some particularly preferred embodiments, the compounds described herein include the polyalkylene oxide having an average molecular weight of from about 12,000 to about 20,000 daltons or from about 30,000 to about 45,000 daltons. In one particular embodiment, polymeric portion has a molecular weight of about 12,000 or 40,000 daltons.

The polyalkylene oxide includes polyethylene glycols and polypropylene glycols. More preferably, the polyalkylene oxide includes polyethylene glycol (PEG). PEG is generally represented by the structure:

—O—(CH₂C₂O)ₙ—

where (n) represents the degree of polymerization for the polymer, and is dependent on the molecular weight of the polymer. Alternatively, the polyethylene glycol (PEG) residue portion of the invention can be selected from among:

—Y₇₁—(CH₂CH₂O)ₙ—CH₂CH₂Y₇₁—,

—Y₇₁—(CH₂CH₂O)ₙ—CH₂C(═Y₇₂)—Y₇₁—,

—Y₇₁—C(═Y₇₂)—(CH₂)ₐ₇₁—Y₇₃—(CH₂CH₂O)ₙ—CH₂CH₂—Y₇₃—(CH₂)ₐ₇₁—C(═Y₇₂)—Y₇₁—, and

—Y₇₁—(CR₇₁R₇₂)ₐ₇₂—Y₇₃—(CH₂)_{b71}—O—(CH₂CH₂O)ₙ—(CH₂)_{b71}—Y₇₃—(CR₇₁R₇₂)ₐ₇₂—Y₇₁—,

wherein:

Y₇₁ and Y₇₃ are independently O, S, SO, SO₂, NR₇₃ or a bond;

Y₇₂ is O, S, or NR₇₄;

R₇₁₋₇₄ are independently the same moieties which can be used for R₂;

(a71), (a72), and (b71) are independently zero or a positive integer, preferably 0-6, and more preferably 1; and

(n) is an integer from about 10 to about 2300.

Branched or U-PEG derivatives are described in U.S. Pat. Nos. 5,643,575, 5,919,455, 6,113,906 and 6,566,506, the disclosure of each of which is incorporated herein by reference. A non-limiting list of such polymers corresponds to polymer systems (i)-(vii) with the following structures:

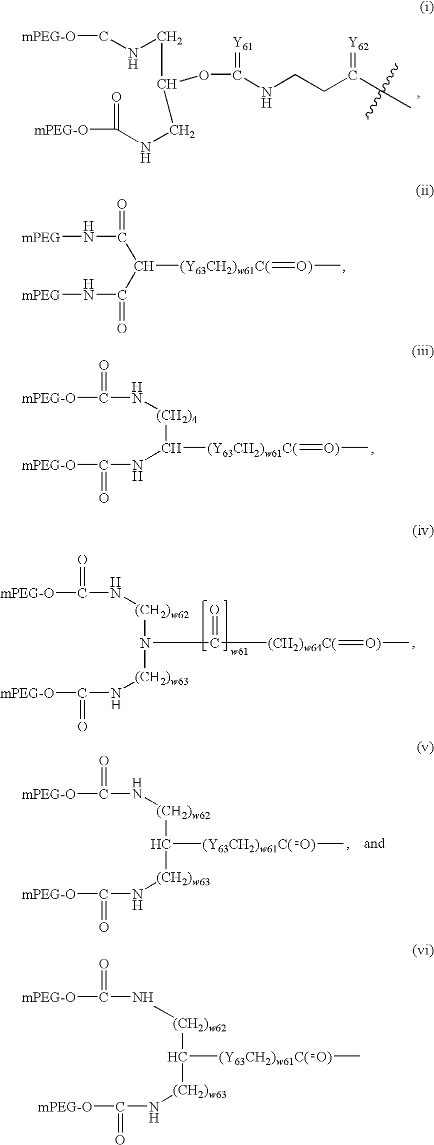

wherein:

Y₆₁₋₆₂ are independently O, S or NR₆₁;

Y₆₃ is O, NR₆₂, S, SO or SO₂

(w62), (w63) and (w64) are independently 0 or a positive integer;

(w61) is 0 or 1;

mPEG is methoxy PEG
wherein PEG is previously defined and a total molecular weight of the polymer portion is from about 2,000 to about 100,000 daltons; and

R₆₁ and R₆₂ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, heteroalkyl, substituted C₁₋₆heteroalkyl, C₁₋₆alkoxy, aryloxy, C₁₋₆heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, and substituted and arylcarbonyloxy.

In yet another aspect, the polymers include multi-arm PEG-OH or “star-PEG” products such as those described in NOF Corp. Drug Delivery System catalog, Ver. 8, April 2006, the disclosure of which is incorporated herein by reference. The polymers can be converted into suitably activated forms, using the activation techniques described in U.S. Pat. Nos. 5,122,614 or 5,808,096 patents. Specifically, such PEG can be of the formula:

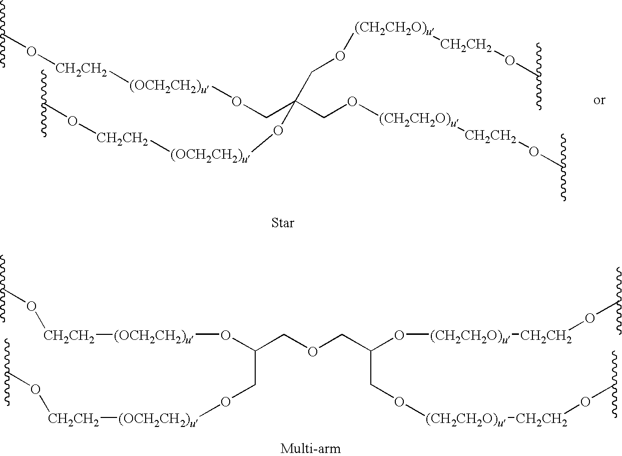

wherein:

(u′) is an integer from about 4 to about 455; and up to 3 terminal portions of the residue is/are capped with a methyl or other lower alkyl.

In some preferred embodiments, all 4 of the PEG arms can be converted to suitable activating groups, for facilitating attachment to aromatic groups. Such compounds prior to conversion include:

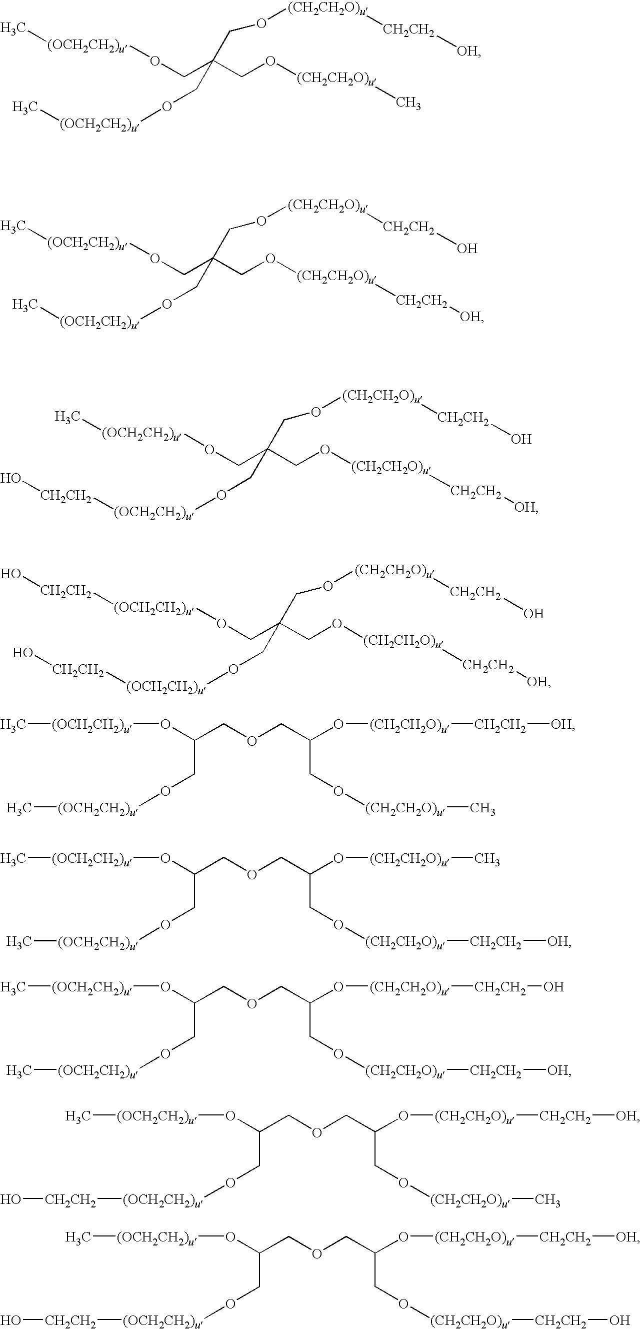

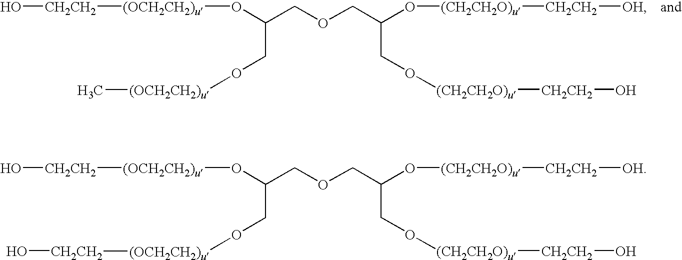

The polymeric substances included herein are preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In a further embodiment and as an alternative to PAO-based polymers, one or more effectively non-antigenic materials such as dextran, polyvinyl alcohols, carbohydrate-based polymers, hydroxypropylmethacrylamide polyalkylene oxides, and/or copolymers thereof can be used. See also commonly-assigned U.S. Pat. No. 6,153,655, the contents of which are incorporated herein by reference. It will be understood by those of ordinary skill that the same type of activation is employed as described herein as for PAO's such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

In some aspects, polymers having terminal amine groups can be employed to make the compounds described herein. The methods of preparing polymers containing terminal amines in high purity are described in U.S. patent application Ser. Nos. 11/508,507 and 11/537,172, the contents of each of which are incorporated by reference. For example, polymers having azides react with phosphine-based reducing agent such as triphenylphosphine or an alkali metal borohydride reducing agent such as NaBH₄. Alternatively, polymers including leaving groups react with protected amine salts such as potassium salt of methyl-tert-butyl imidodicarbonate (KNMeBoc) or the potassium salt of di-tert-butyl imidodicarbonate (KNBoc₂) followed by deprotecting the protected amine group. The purity of the polymers containing the terminal amines formed by these processes is greater than about 95% and preferably greater than 99%.

In alternative aspects, polymers having terminal carboxylic acid groups can be employed in the polymeric delivery systems described herein. Methods of preparing polymers having terminal carboxylic acids in high purity are described in U.S. patent application Ser. No. 11/328,662, the contents of which are incorporated herein by reference. The methods include first preparing a tertiary alkyl ester of a polyalkylene oxide followed by conversion to the carboxylic acid derivative thereof. The first step of the preparation of the PAO carboxylic acids of the process includes forming an intermediate such as t-butyl ester of polyalkylene oxide carboxylic acid. This intermediate is formed by reacting a PAO with a t-butyl haloacetate in the presence of a base such as potassium t-butoxide. Once the t-butyl ester intermediate has been formed, the carboxylic acid derivative of the polyalkylene oxide can be readily provided in purities exceeding 92%, preferably exceeding 97%, more preferably exceeding 99% and most preferably exceeding 99.5% purity.

### C. Bifunctional Linkers

Bifunctional linkers include amino acids, amino acid derivatives, and peptides. The amino acids can be among naturally occurring and non-naturally occurring amino acids. Derivatives and analogs of the naturally occurring amino acids, as well as various art-known non-naturally occurring amino acids (D or L), hydrophobic or non-hydrophobic, are also contemplated to be within the scope of the invention. A suitable non-limiting list of the non-naturally occurring amino acids includes 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, alto-isoleucine, N-methylglycine, sarcosine, N-methyl-isoleucine, 6-N-methyl-lysine, N-methylvaline, norvaline, norleucine, and ornithine. Some preferred amino acid residues are selected from glycine, alanine, methionine or sarcosine, and more preferably, glycine.

Alternatively, L₁₋₃ and L′₁₋₃ are independently selected from among:

—[C(═O)]ᵥ(CR₂₂R₂₃)ₜ[C(═O)]_{v′}—,

—[C(═O)]ᵥ(CR₂₂R₂₃)ₜ—O[C(═O)]_{v′}—,

—[C(═O)]ᵥ(CR₂₂R₂₃)ₜ—NR₂₆[C(═O)]_{v′}—,

—[C(═O)]ᵥO(CR₂₂R₂₃)ₜ[C(═O)]_{v′}—,

—[C(═O)]ᵥO(CR₂₂R₂₃)ₜO[C(═O)]_{v′}—,

—[C(═O)]ᵥO(CR₂₂R₂₃)ₜNR₂₆[C(═O)]_{v′}—,

—[C(═O)]ᵥNR₂₁(CR₂₂R₂₃)ₜ[C(═O)]_{v′}—,

—[C(═O)]ᵥNR₂₁(CR₂₂R₂₃)ₜO[C(═O)]_{v′}—,

—[C(═O)]ᵥNR₂₁(CR₂₂R₂₃)ₜNR₂₆[C(═O)]_{v′}—,

—[C(═O)]ᵥ(CR₂₂R₂₃)ₜO—(CR₂₈R₂₉)_{t′}[C(═O)]_{v′}—,

—[C(═O)]ᵥ(CR₂₂R₂₃)ₜNR₂₆—(CR₂₈R₂₉)_{t′}[C(═O)]_{v′}—,

—[C(═O)]ᵥ(CR₂₂R₂₃)ₜS—(CR₂₈R₂₉)_{t′}[C(═O)]_{v′}—,

—[C(═O)]ᵥO(CR₂₂R₂₃)ₜO—(CR₂₈R₂₉)_{t′}[C(═O)]_{v′}—,

—[C(═O)]ᵥO(CR₂₂R₂₃)ₜNR₂₆—(CR₂₈R₂₉)_{t′}[C(═O)]_{v′}—,

—[C(═O)]ᵥO(CR₂₂R₂₃)ₜS—(CR₂₈R₂₉)_{t′}[C(═O)]_{v′}—,

—[C(═O)]ᵥNR₂₁(CR₂₂R₂₃)ₜO—(CR₂₈R₂₉)_{t′}[C(═O)]_{v′}—,

—[C(═O)]ᵥNR₂₁(CR₂₂R₂₃)ₜNR₂₆—(CR₂₈R₂₉)_{t′}[C(═O)]_{v′}—,

—[C(═O)]ᵥNR₂₁(CR₂₂R₂₃)ₜS—(CR₂₈R₂₉)_{t′}[C(═O)]_{v′}—,

—[C(═O)]ᵥ(CR₂₂R₂₃CR₂₈R₂₉O)ₜNR₂₆[C(═O)]_{v′}—,

—[C(═O)]ᵥ(CR₂₂R₂₃CR₂₈R₂₉O)ₜ[C(═O)]_{v′}—,

—[C(═O)]ᵥO(CR₂₂R₂₃CR₂₈R₂₉O)ₜNR₂₆[C(═O)]_{v′}—,

—[C(═O)]ᵥO(CR₂₂R₂₃CR₂₈R₂₉O)ₜ[C(═O)]_{v′}—,

—[C(═O)]ᵥNR₂₁(CR₂₂R₂₃CR₂₈R₂₉O)ₜNR₂₆[C(═O)]_{v′}—,

—[C(═O)]ᵥNR₂₁(CR₂₂R₂₃CR₂₈R₂₉O)ₜ[C(═O)]_{v′}—,

—[C(═O)]ᵥ(CR₂₂R₂₃CR₂₈R₂₉O)ₜ(CR₂₄R₂₅)_{t′}[C(═O)]_{v′}—,

—[C(═O)]ᵥO(CR₂₂R₂₃CR₂₈R₂₉O)ₜ(CR₂₄R₂₅)_{t′}[C(═O)]_{v′}—,

—[C(═O)]ᵥNR₂₁(CR₂₂R₂₃CR₂₈R₂₉O)ₜ(CR₂₄R₂₅)_{t′}[C(═O)]_{v′}—,

—[C(═O)]ᵥ(CR₂₂R₂₃CR₂₈R₂₉O)ₜ(CR₂₄R₂₅)_{t′}O[C(═O)]_{v′}—,

—[C(═O)]ᵥ(CR₂₂R₂₃)ₜ(CR₂₄R₂₅CR₂₈R₂₉O)_{t′}[C(═O)]_{v′}—,

—[C(═O)]ᵥ(CR₂₂R₂₃)ₜ(CR₂₄R₂₅CR₂₈R₂₉O)_{t′}NR₂₆[C(═O)]_{v′}—,

—[C(═O)]ᵥO(CR₂₂R₂₃CR₂₈R₂₉O)ₜ(CR₂₄R₂₅)_{t′}O[C(═O)]_{v′}—,

—[C(═O)]ᵥO(CR₂₂R₂₃)ₜ(CR₂₄R₂₅CR₂₈R₂₉O)_{t′}[C(═O)]_{v′}—,

—[C(═O)]ᵥO(CR₂₂R₂₃)ₜ(CR₂₄R₂₅CR₂₈R₂₉O)_{t′}NR₂₆[C(═O)]_{v′}—,

—[C(═O)]ᵥNR₂₁(CR₂₂R₂₃CR₂₈R₂₉O)ₜ(CR₂₄R₂₅)_{t′}O[C(═O)]_{v′}—,

—[C(═O)]ᵥNR₂₁(CR₂₂R₂₃)ₜ(CR₂₄R₂₅CR₂₈R₂₉O)_{t′}[C(═O)]_{v′}—,

—[C(═O)]ᵥNR₂₁(CR₂₂R₂₃)ₜ(CR₂₄R₂₅CR₂₈R₂₉O)_{t′}NR₂₆[C(═O)]_{v′}—,

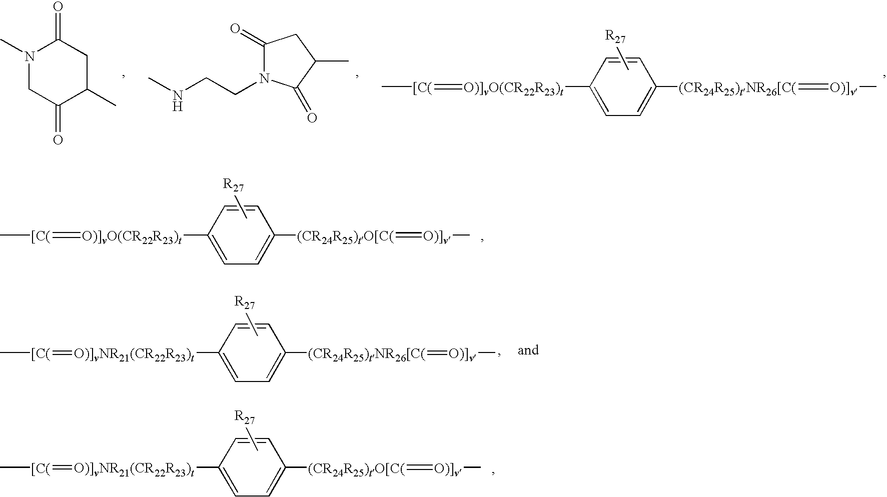

wherein:

R₂₁₋₂₉ are independently selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;

(t) and (t′) are independently zero or a positive integer, preferably zero or an integer from about 1 to about 12, more preferably an integer from about 1 to about 8, and most preferably 1 or 2; and

(v) and (v′) are independently zero or 1.

In some preferred embodiments, L₁₋₃ and L′₁₋₃ are independently selected from among:

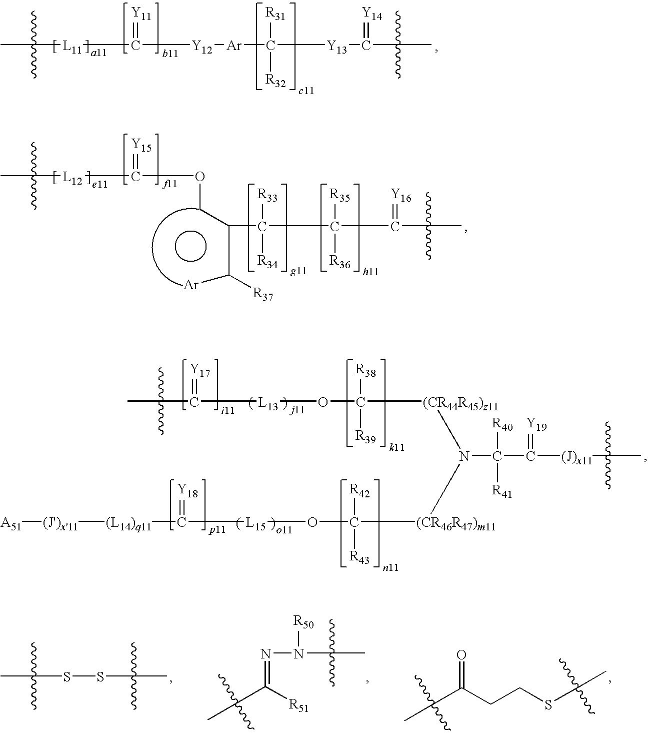

-Val-Cit-,

-Gly-Phe-Leu-Gly-,

-Ala-Leu-Ala-Leu-,

-Phe-Lys-,

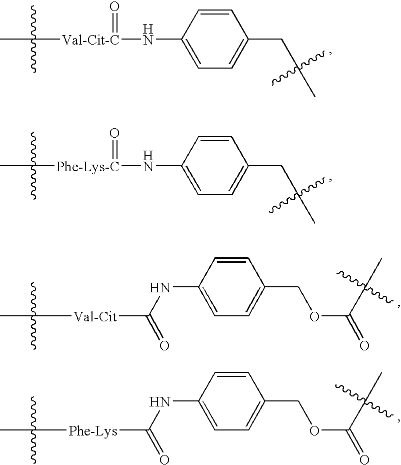

-Val-Cit-C(═O)—CH₂OCH₂—C(═O)—,

-Val-Cit-C(═O)—CH₂SCH₂—C(═O)—, and

—NHCH(CH₃)—C(═O)—NH(CH₂)₆—C(CH₃)₂—C(═O)—

wherein,

Y₁₁₋₁₉ are independently O, S or NR₄₈;

R₃₁₋₄₈, R₅₀₋₅₁ and A₅₁ are independently selected from among hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆heteroalkyls, C₁₋₆ alkoxy, phenoxy and C₁₋₆ heteroalkoxy;

Ar is an aryl or heteroaryl moiety;

L₁₁₋₁₅ are independently selected bifunctional spacers;

J and J′ are independently selected from selected from among moieties actively transported into a target cell, hydrophobic moieties, bifunctional linking moieties and combinations thereof;

(c11), (h11), (k11), (z11), (m11) and (n11) are independently selected positive integers, preferably 1;

(a11), (e11), (g11), (j11), (o11) and (q11) are independently either zero or a positive integer, preferably 1; and

(b11), (x11), (x′11), (f11), (i11) and (p11) are independently zero or one.

Alternatively, L₁₋₃ and L′₁₋₃ are independently selected from among:

—[C(═O)]ᵣNH(CH₂)₂CH═N—NHC(═O)—(CH₂)₂—,

—[C(═O)]ᵣNH(CH₂)₂(CH₂CH₂O)₂(CH₂)₂NH[C(═O)]_{r′}—,

—[C(═O)]ᵣNH(CH₂CH₂)(CH₂CH₂O)₂NH[C(═O)]_{r′}—,

—[C(═O)]ᵣNH(CH₂CH₂)ₛNH(CH₂CH₂)_{s′}[C(═O)]_{r′}—,

—[C(═O)]ᵣNH(CH₂CH₂)ₛS(CH₂CH₂)_{s′}[C(═O)]_{r′}—,

—[C(═O)]ᵣNH(CH₂CH₂)(CH₂CH₂O)[C(═O)]_{r′}—,

—[C(═O)]ᵣNH(CH₂CH₂)ₛO(CH₂CH₂)_{s′}[C(═O)]_{r′}—,

—[C(═O)]ᵣNH(CH₂CH₂O)(CH₂CH₂)NH[C(═O)]_{r′}—,

—[C(═O)]ᵣNH(CH₂CH₂O)₂(CH₂)[C(═O)]_{r′}—,

—[C(═O)]ᵣNH(CH₂CH₂O)ₛ(CH₂)_{s′}[C(═O)]_{r′}—,

—[C(═O)]ᵣNHCH₂CH₂NH[C(═O)]_{r′}—,

—[C(═O)]ᵣNH(CH₂CH₂)₂O[C(═O)]_{r′}—,

—[C(═O)]ᵣNH(CH₂CH₂O)[C(═O)]_{r′}—,

—[C(═O)]ᵣNH(CH₂CH₂O)₂[C(═O)]_{r′}—,

—[C(═O)]ᵣNH(CH₂)₃[C(═O)]_{r′}—,

—[C(═O)]ᵣO(CH₂CH₂O)₂(CH₂)[C(═O)]_{r′}—,

—[C(═O)]ᵣO(CH₂)₂NH(CH₂)₂[C(═O)]_{r′}—,

—[C(═O)]ᵣO(CH₂CH₂O)₂NH[C(═O)]_{r′}—,

—[C(═O)]ᵣO(CH₂)₂O(CH₂)₂[C(═O)]_{r′}—,

—[C(═O)]ᵣO(CH₂)₂S(CH₂)₂[C(═O)]_{r′}—,

—[C(═O)]ᵣO(CH₂CH₂)NH[C(═O)]_{r′}—,

—[C(═O)]ᵣO(CH₂CH₂)O[C(═O)]_{r′}—,

—[C(═O)]ᵣO(CH₂)₃NH[C(═O)]_{r′}—,

—[C(═O)]ᵣO(CH₂)₃O[C(═O)]_{r′}—,

—[C(═O)]ᵣO(CH₂)₃[C(═O)]_{r′}—,

—[C(═O)]ᵣCH₂NHCH₂[C(═O)]_{r′}—,

—[C(═O)]ᵣCH₂OCH₂[C(═O)]_{r′}—,

—[C(═O)]ᵣCH₂SCH₂[C(═O)]_{r′}—,

—[C(═O)]ᵣS(CH₂)₃[C(═O)]_{r′}—,

—[C(═O)]ᵣ(CH₂)₃[C(═O)]_{r′}—,

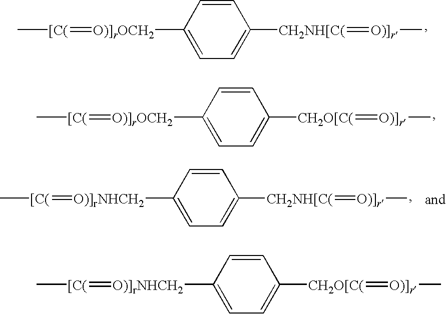

wherein, (r) and (r′) are independently zero or 1.

In a further embodiment and as an alternative, L₁₋₃ and L′₁₋₃ include structures corresponding to those shown above but substituted further with vinyl, residues of sulfone, amino, carboxy, mercapto, hydrazide, carbazate and the hie.

### D. R₁₂ and R′₁₂ Groups

### 1. Leaving Groups and Functional Groups

In some aspects, suitable leaving groups include, without limitations halogen (Br, Cl), activated carbonate, carbonyl imidazole, cyclic imide thione, isocyanate, N-hydroxysuccinimidyl, para-nitrophenoxy, N-hydroxyphtalimide, N-hydroxybenzotriazolyl, imidazole, tosylate, mesylate, tosylate, nosylate, C₁-C₆ alkyloxy, C₁-C₆ alkanoyloxy, arylcarbonyloxy, ortho-nitrophenoxy, N-hydroxybenzotriazolyl, imidazole, pentafluorophenoxy, 1,3,5-trichlorophenoxy, and 1,3,5-trifluorophenoxy or other suitable leaving groups as will be apparent to those of ordinary skill.

For purposes of the present invention, leaving groups are to be understood as those groups which are capable of reacting with a nucleophile found on the desired target, i.e. a biologically active moiety, a diagnostic agent, a targeting moiety, a bifunctional spacer, intermediate, etc. The targets thus contain a group for displacement, such as OH, NH₂ or SH groups found on proteins, peptides, enzymes, naturally or chemically synthesized therapeutic molecules such as doxorubicin, and spacers such as mono-protected diamines.

In some preferred embodiments, functional groups to link the polymeric transport systems to biologically active moieties include maleimidyl, vinyl, residues of sulfone, amino, carboxy, mercapto, hydrazide, carbazate and the like which can be further conjugated to a biologically active group.

In yet some preferred embodiments of the invention, R₁₂ and R′₁₂ can be selected from among H, OH, methoxy, tert-butoxy, N-hydroxysuccinimidyl and maleimidyl.

### 2. Biologically Active Moieties

A wide variety of biologically active moieties can be attached to the activated polymers described herein. The biologically active moieties include pharmaceutically active compounds, enzymes, proteins, oligonucleotides, antibodies, monoclonal antibodies, single chain antibodies and peptides. A biologically active compound to conjugate with the compound in the invention will contain SH functional moiety. In addition, the activated polymer of the invention can further contain a biologically active moiety as R₁₂ which includes amine-, hydroxyl-, or thiol-containing compounds. A non-limiting list of such suitable compounds includes organic compounds, enzymes, proteins, polypeptides, antibodies, monoclonal antibodies, single chain antibodies or oligonucleotides, etc. Organic compounds include, without limitation, moieties such as camptothecin and analogs such as SN38, irinotecan, and related topoisomerase I inhibitors, taxanes and paclitaxel derivatives, nucleosides including AZT, anthracycline compounds including daunorubicin, doxorubicin; p-aminoaniline mustard, melphalan, Ara-C (cytosine arabinoside) and related anti-metabolite compounds, e.g., gemcitabine, etc. Alternatively, biologically active moieties can include cardiovascular agents, anti-neoplastic, anti-infective, anti-fungal such as nystatin and amphotericin B, anti-anxiety agents, gastrointestinal agents, central nervous system-activating agents, analgesic, fertility agents, contraceptive agents, anti-inflammatory agents, steroidal agents, anti-ureceroic agents, vasodilating agents, and vasoconstricting agents, etc. It is to be understood that other biologically active materials not specifically mentioned but having suitable amine-, hydroxyl- or thiol-containing groups are also intended and are within the scope of the present invention.

In another aspect of the invention, the biologically, active compounds are suitable for medicinal or diagnostic use in the treatment of animals, e.g., mammals, including humans, for conditions for which such treatment is desired.

The only limitations on the types of the biologically active moieties suitable for inclusion herein is that there is available at least one chemically reactive functional moiety such as amine, hydroxyl, or thiol to link with a carrier portion and that there is not substantial loss of bioactivity in the form conjugated to the polymeric delivery systems described herein. Alternatively, parent compounds suitable for incorporation into the polymeric transport conjugate compounds of the invention, may be active after hydrolytic release from the linked compound, or not active after hydrolytic release but which will become active after undergoing a further chemical process/reaction. For example, an anticancer drug that is delivered to the bloodstream by the polymeric transport system, may remain inactive until entering a cancer or tumor cell, whereupon it is activated by the cancer or tumor cell chemistry, e.g., by an enzymatic reaction unique to that cell.

A further aspect of the invention provides the conjugate compounds optionally prepared with a diagnostic tag linked to the polymeric delivery system described herein, wherein the tag is selected for diagnostic or imaging purposes. Thus, a suitable tag is prepared by linking any suitable moiety, e.g., an amino acid residue, to any art-standard emitting isotope, radio-opaque label, magnetic resonance label, or other non-radioactive isotopic labels suitable for magnetic resonance imaging, fluorescence-type labels, labels exhibiting visible colors and/or capable of fluorescing under ultraviolet, infrared or electrochemical stimulation, to allow for imaging tumor tissue during surgical procedures, and so forth. Optionally, the diagnostic tag is incorporated into and/or linked to a conjugated therapeutic moiety, allowing for monitoring of the distribution of a therapeutic biologically active material within an animal or human patient.

In a still further aspect of the invention, the inventive tagged conjugates are readily prepared, by art-known methods, with any suitable label, including, e.g., radioisotope labels. Simply by way of example, these include ¹³¹Iodine, ¹²⁵Iodine, ^{99m}Technetium and/or ¹¹¹Indium to produce radioimmuno-scintigraphic agents for selective uptake into tumor cells, in vivo. For instance, there are a number of art-known methods of linking peptide to Tc-99m, including, simply by way of example, those shown by U.S. Pat. Nos. 5,328,679; 5,888,474; 5,997,844; and 5,997,845, incorporated by reference herein.

### 3. Targeting Groups

In some aspects, the compounds described herein can react with or contain targeting groups. The targeting groups include receptor ligands, an antibodies or antibody fragments, single chain antibodies, targeting peptides, targeting carbohydrate molecules or lectins. Targeting groups enhance binding or uptake of the compounds described herein a target tissue and cell population. For example, a non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides. PD1 and PD1 peptides, EGF and EGF peptides, RGD peptides, folate, etc. In another aspect of the invention the targeting groups include monoclonal antibody, single chain antibody, biotin, cell adhesion peptides, cell penetrating peptides (CPPs), fluorescent compounds, radio-labeled compounds, and aptamers. In a still further aspect of the invention the targeting agent can include Selectin, TAT, Penetratin, Ang9, and folic acid.

### E. Synthesis of the Polymeric Delivery Systems

Generally, the methods of preparing the activated polymer of the invention include reacting the polymer with a proper leaving group with a nucleophile containing pyridyl disulfide group at the distal end. The activated polymer delivery system of the invention can further react with a biologically active compound containing SH group to provide the polymeric conjugate where the biologically active moiety is bonded to the polymer through —S—S— bond.

In one aspect of the invention, methods of preparing compounds described herein include:

reacting a polymeric compound of Formula (III):

A₄-R₁-M₁ (III)

with a compound of Formula (IV):

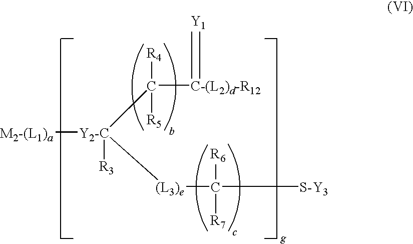

under conditions sufficient to form a compound of the formula (V):

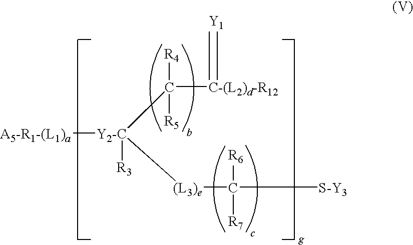

wherein:

R₁ is a substantially non-antigenic water-soluble polymer,

A₄ is a capping group or M₁;

A₅ is a capping group or

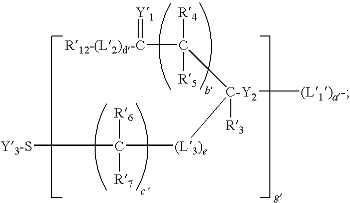

M₁ is OH or a leaving group;

M₂ is —OH, SH, or —NHR₉₀;

Y₁ and Y′₁ are independently S, O, or NR₂;

Y₂ and Y′₂ are independently S, O, SO, SO₂, NR₂₀;

Y₃ and Y′₃ are independently H, leaving group, activating group, functional group, or

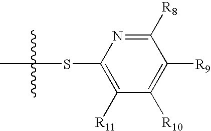

L₁₋₃ and L′₁₋₃ are independently selected bifunctional linkers;

R₂₋₁₁, R′₂₋₁₁, R₂₀ and R₉₀ are independently selected from among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;

R₁₂ and R′₁₂ are independently selected from among hydrogen, hydroxyl, leaving group, functional group, medicinal agent, targeting agent, diagnostic agent, substituted. C₁₋₆ alkylthio, C₁₋₆alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆heteroalkyl, C₁₋₆alkoxy, aryloxy, C₁₋₆heteroalkoxy, heteroaryloxy, C₂₋₆ alkanoyl, arylcarbonyl, C₂₋₆ alkoxycarbonyl aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted arylcarbonyloxy, malcimidyl, vinyl, substituted sulfone, amino, carboxy, mercapto, hydrazide and carbazate;

(a), (a′), (d) and (d′) are independently zero or a positive integer;

(b) and (b′) are independently zero or a positive integer;

(c) and (c′) are independently zero or a positive integer;

(e) and (e′) are independently zero or 1; and

(g) and (g′) are independently zero or 1;

provided that (a) and (g) are not simultaneously zero.

Attachment of the pyridyl disulfide containing moiety to the polymer portion or conjugation of the polymeric system containing branching moiety with the compound of Formula (IV) is preferably carried out in the presence of a coupling agent. A non-limiting list of suitable coupling agents include 1,3-diisopropylcarbodiimide (DIPC), any suitable dialkyl carbodiimides, 2-halo-1-alkyl-pyridinium halides, (Mukaiyama reagents), 1-(3-dimethylaminopropyl)-3-ethyl carbodiimide (EDC), propane phosphonic acid cyclic anhydride (PPACA), and phenyl dichlorophosphates, etc. which are available, for example from commercial sources such as Sigma-Aldrich Co., or synthesized using known techniques.

Preferably, the reactions are carried out in an inert solvent such as methylene chloride, chloroform, DMF or mixtures thereof. The reactions can be preferably conducted in the presence of a base, such as dimethylaminopyridine (DMAP), diisopropylethylamine, pyridine, triethylamine, etc. to neutralize any acids generated. The reactions can be carried out at a temperature from about 0° C. up to about 22° C. (room temperature).

Some particular embodiments prepared by the methods described herein include:

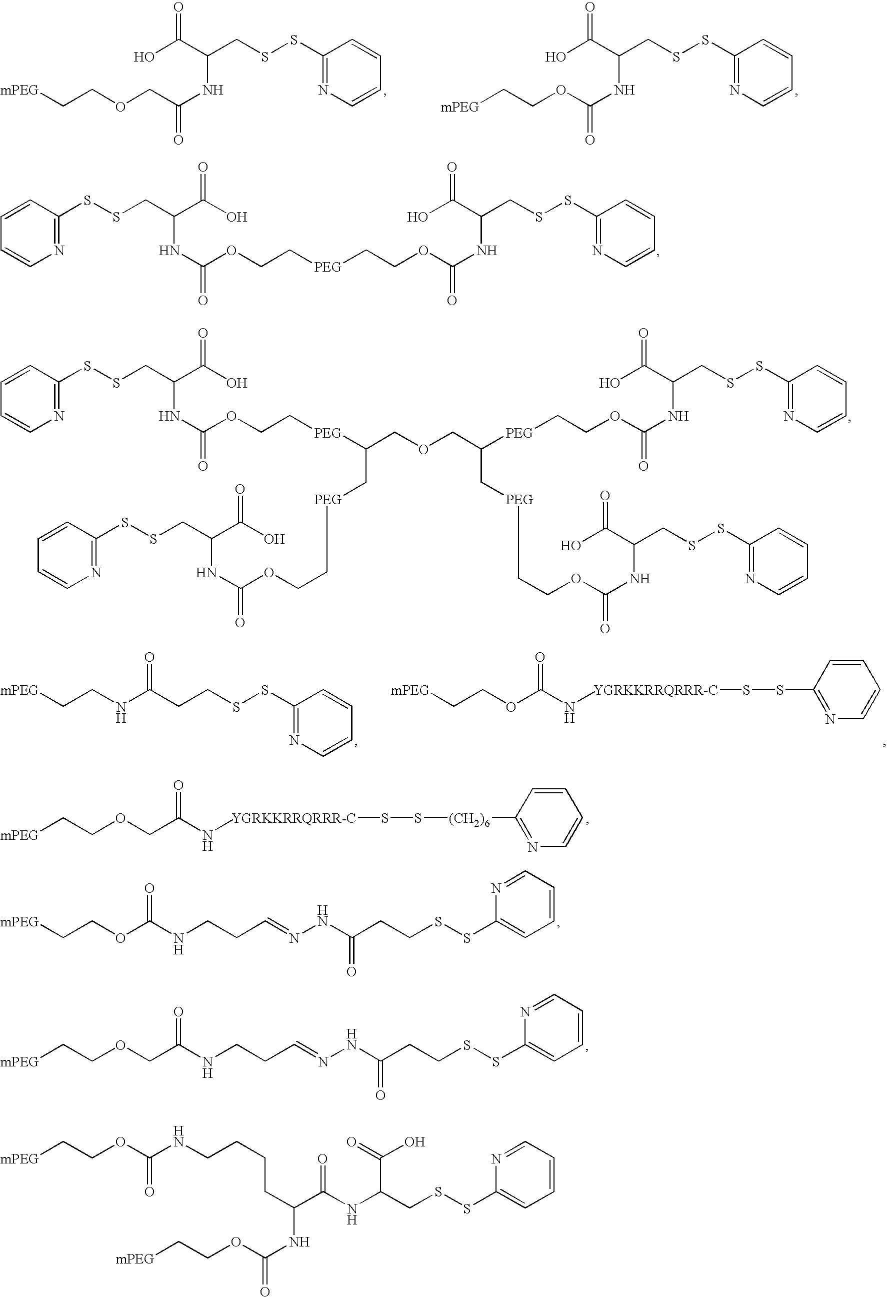

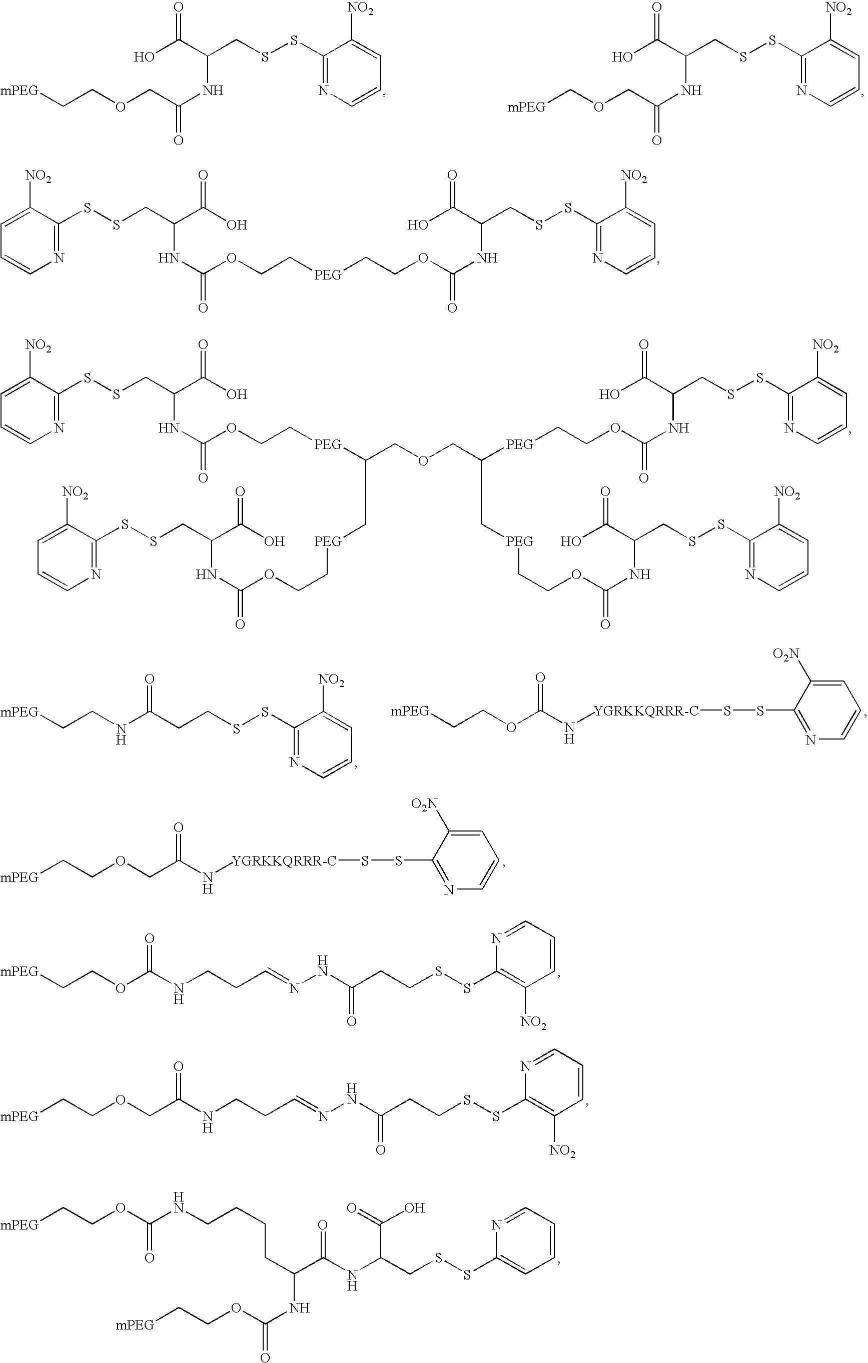

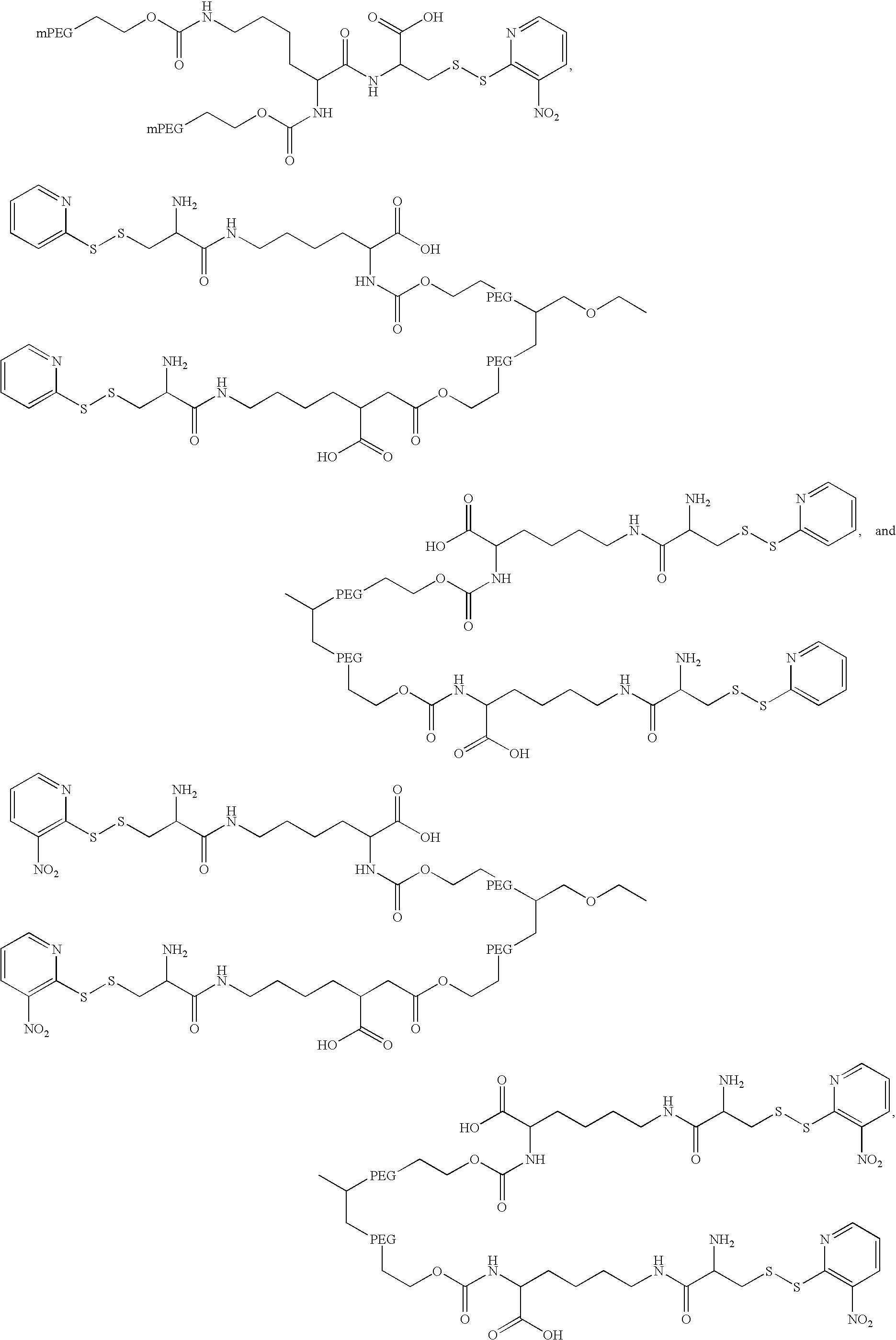

wherein:

mPEG has the formula CH₃O(CH₂CH₂O)ₙ—;

PEG has the formula —O(CH₂CH₂O)ₙ—, and

(n) is an integer from about 10 to about 2,300.

The resulting compound of Formula (V) can further react with a SH containing moiety to provide a polymeric delivery conjugate with a biologically moiety bonded via disulfide bond. The activated polymer of the invention can readily conjugate with a biologically active moiety in a neutral or a mild acidic condition such as pH 6.5. The reaction can be run at room temperature or −4° C. to 30° C. in a solvent suitable for polymeric compound of the invention and the biologically active moiety. The reaction can be run either in aqueous or organic solvent such as DCM, chloroform, DMF, DMSO, etc. It would be preferable to run the reaction in aqueous buffer solution if the substrate is oligonucleotides or peptides. The biologically active moiety is selected from among pharmaceutically active compounds, enzymes, proteins, oligonucleotides, antibodies, monoclonal antibodies, single chain antibodies and peptides. Some methods of conjugation are described in the examples.

Some particular embodiments which can be prepared by reacting the activated polymeric compound of the invention with a biologically active moiety using the conjugation methods described herein include:

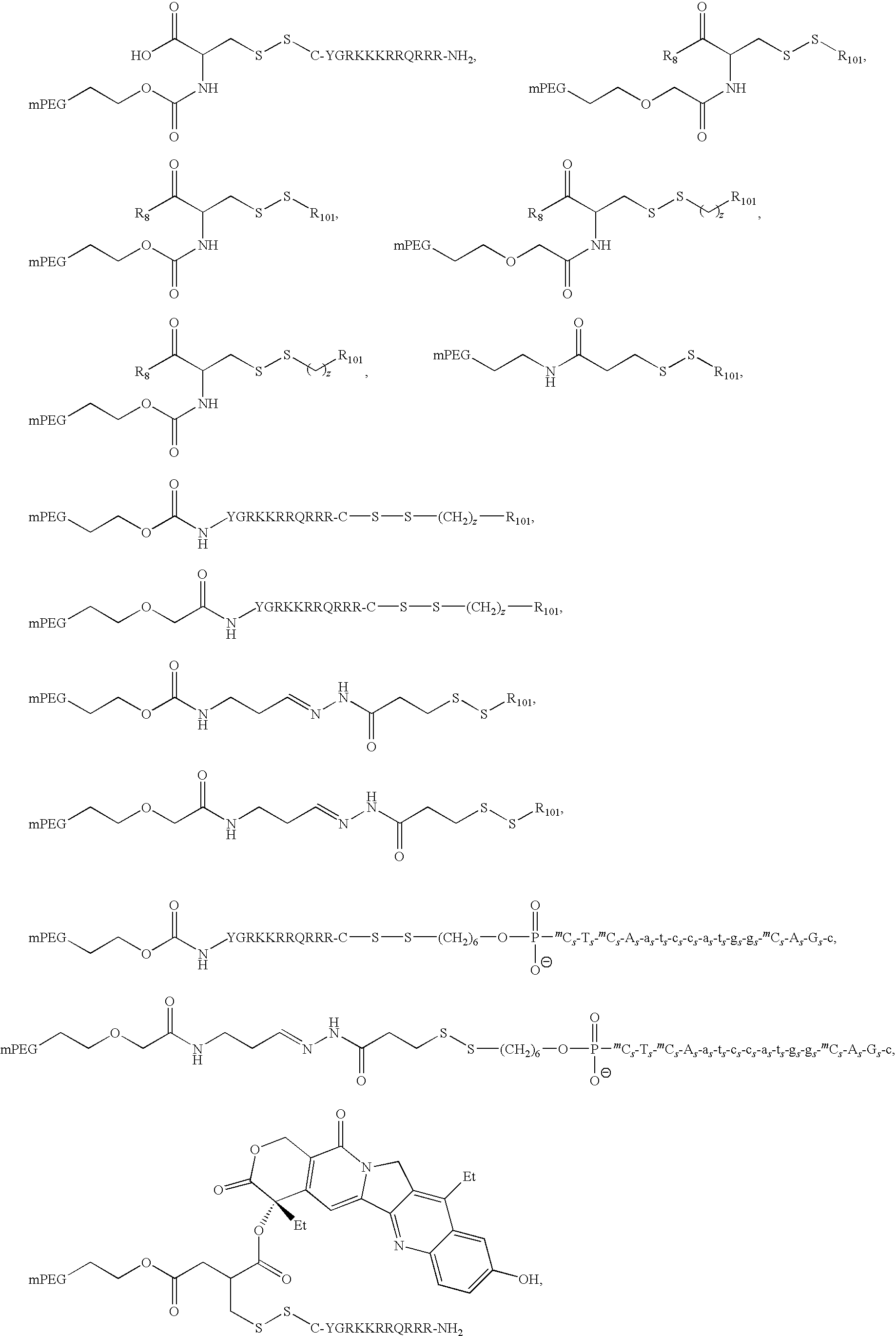

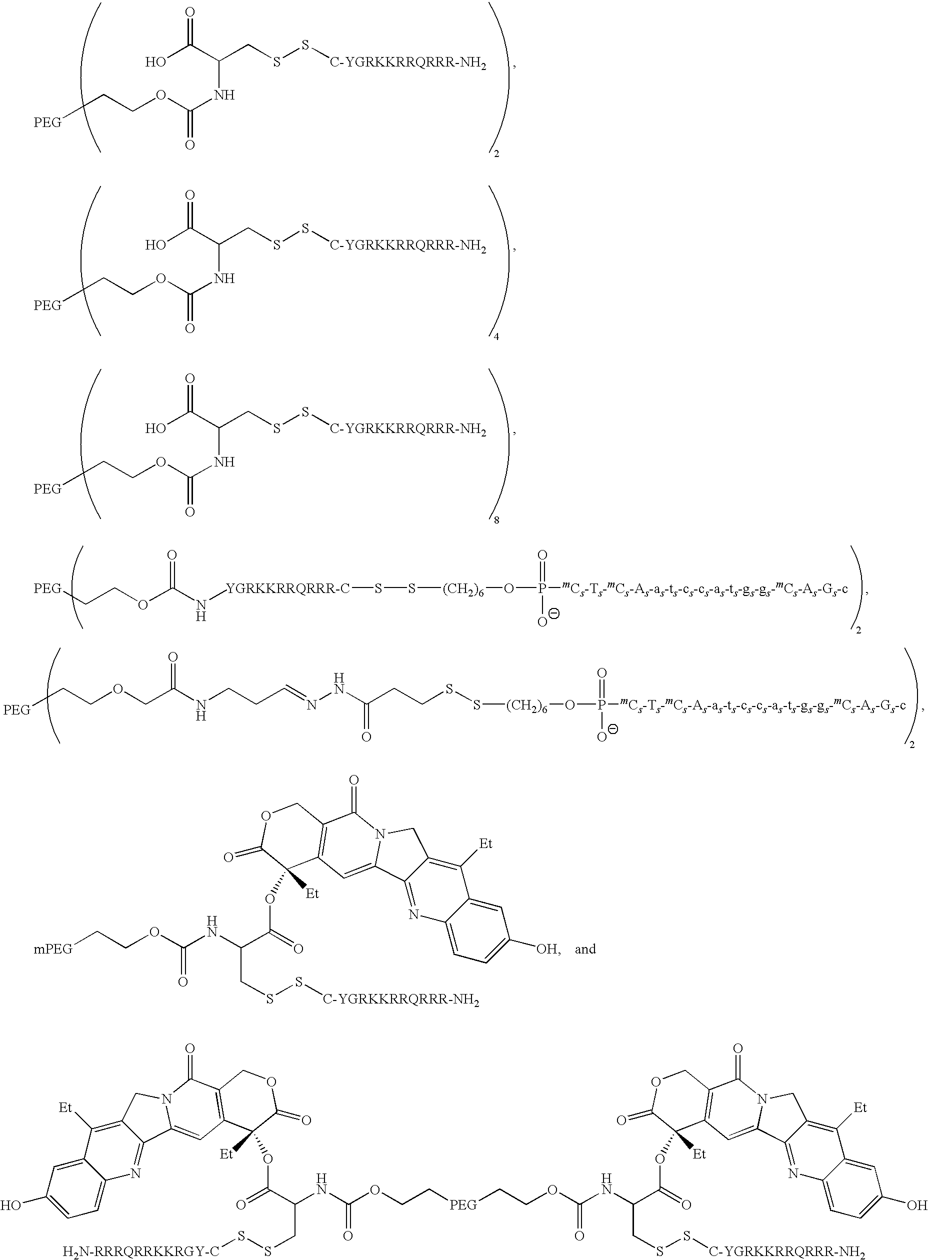

wherein:

(z) is a positive integer, preferably from about 1 to about 10;

—YGRKKRRQRRR— is TAT peptide;

mPEG has the formula CH₃O(CH₂CH₂O)ₙ—;

PEG has the formula —O(CH₂CH₂O)ₙ—,

(n) is an integer from about 10 to about 2,300; and

R₁₀₁ is selected from among targeting groups, diagnostic agents and biologically active moieties.

### F. Methods of Treatment

Another aspect of the present invention provides methods of treatment for various medical conditions in mammals. The methods include administering, to the mammal in need of such treatment, an effective amount of a biologically active moiety conjugated polymer, described herein. The polymeric conjugate compounds are useful for, among other things, treating diseases which are similar to those which are treated with the parent compound, e.g. enzyme replacement therapy, neoplastic disease, reducing tumor burden, preventing metastasis of neoplasms and preventing recurrences of tumor/neoplastic growths in mammals.

The amount of the polymeric conjugate that is administered will depend upon the amount of the parent molecule included therein. Generally, the amount of polymeric conjugate used in the treatment methods is that amount which effectively achieves the desired therapeutic result in mammals. Naturally, the dosages of the various polymeric conjugate compounds will vary somewhat depending upon the parent compound, molecular weight of the polymer, rate of in vivo hydrolysis, etc. Those skilled in the art will determine the optimal dosing of the polymeric transport conjugates selected based on clinical experience and the treatment indication. Actual dosages will be apparent to the artisan without undue experimentation.

The compounds of the present invention can be included in one or more suitable pharmaceutical compositions for administration to mammals. The pharmaceutical compositions may be in the form of a solution, suspension, tablet, capsule or the like, prepared according to methods well known in the art. It is also contemplated that administration of such compositions may be by the oral and/or parenteral routes depending upon the needs of the artisan. A solution and/or suspension of the composition may be utilized, for example, as a carrier vehicle for injection or infiltration of the composition by any art known methods, e.g., by intravenous, intramuscular, intraperitoneal, subcutaneous injection and the like. Such administration may also be by infusion into a body space or cavity, as well as by inhalation and/or intranasal routes. In preferred aspects of the invention, however, the polymeric conjugates are parenterally administered to mammals in need thereof.

### EXAMPLES

The following examples serve to provide further appreciation of the invention but are not meant in any way to restrict the scope of the invention. The bold-faced numbers recited in the Examples correspond to those shown in Figures. Abbreviations are used throughout the examples such as, DCM (dichloromethane), DIEA (diisopropylethylamine), DMAP (4-dimethylaminopyridine), DMF (N,N′-dimethylformamide), DSC (disuccinimidyl carbonate), EDC (1-(3-dimethylaminopropyl)-3-ethyl carbodiimide), IPA (isopropanol), NHS(N-hydroxysuccinimide), PEG (polyethylene glycol), SCA-SH (single-chain antibody), SN38 (7-ethyl-10-hydroxycamptothecin), TBDPS (tert-butyl-dipropylsilyl), and TEA (triethylamine).

General Procedures. All reactions are run under an atmosphere of dry nitrogen or argon. Commercial reagents are used without further purification. All PEG compounds are dried in vacuo or by azeotropic distillation from toluene prior to use. ¹H NMR spectra were obtained at 300 MHz and ¹³C NMR spectra were obtained at 75.46 MHz using a Varian Mercury®300 NMR spectrometer and deuterated chloroform as the solvents unless otherwise specified. Chemical shifts (δ) are reported in parts per million (ppm) downfield from tetramethylsilane (TMS).

HPLC Method. The reaction mixtures and the purity of intermediates and final products are monitored by a Beckman Coulter System Gold® HPLC instrument. It employs a ZORBAX® 300SB C8 reversed phase column (150×4.6 mm) or a Phenomenex Jupiter® 300A C18 reversed phase column (150×4.6 mm) with a 168 Diode Array UV Detector, using a gradient of 10-90% of acetonitrile in 0.05% trifluoroacetic acid (TFA) at a flow rate of 1 mL/min.

### Example 1

### Preparation of Compound (2)

A solution of 4 N HCl in dioxane (70 mL) was added to BocCys(Npys)-OH (compound 1, 1.5 g, 13.32 mmol). The suspension was stirred at room temperature for 3 h, and then was poured into 700 mL of ethyl ether. The solid was collected by gravity filtration using a coarse filter and washed with ethyl ether (50 mL) three times. The washed solid was dried in vacuo at room temperature overnight to give the product: ¹H NMR (CD₃OD) d 8.93 (1H, dd, J=1.5, 4.7 Hz), 8.66 (1H, dd, J=1.5, 8.20 Hz), 7.59 (1H, dd, J=4.7, 8.2 Hz), 4.24 (1H, dd, J=4.1, 9.4 Hz), 3.58 (1H, dd, J=4.1, 14.9 Hz), 3.36 (1H, dd, J=9.4, 15.2 Hz). ¹³C NMR (CD₃OD): d 169.40, 156.27, 154.64, 144.13, 135.246, 123.10, 52.77, 39.27.

### Example 2

### Preparation of Compound (4)

mPEG-SC (compound 3, Mw. 20 kDa, 7.30 g, 0.35 mmol) and DIEA (3 mL, 16.8 mmol) are added to a solution of compound 2 (1.82 g, 5.55 mmol) in mixture of DMF and DCM (25 mL-45 mL). The resulting suspension is stirred at room temperature for 5 hours. The reaction mixture is evaporated in vacuo and then precipitated with DCM-Et₂O at 0° C. The solid is collected by filtration and dissolved in 80 mL of DCM. After addition of 20 mL of 0.1 N HCl, the mixture is stirred for 5 minutes. The organic layer is separated using a separatory funnel and washed with 0.1 N HCl (20 mL) and brine (20 mL). The organic layer is dried over anhydrous MgSO₄, filtered and evaporated in vacuo. The residue is precipitated with DCM/Et₂O at 0° C. The solid was filtered and dried in the vacuum oven at 30° C. for at least 2 h to give the product.

### Example 3

### Preparation of Compound (5)

Compound 4 (0.084 mmol) is added to SCA-SH (0.00027 mmol) in 3 mL of sodium phosphate buffer (0.1 M, pH 7.8) with gentle stirring. The solution is stirred at 30° C. for 30 minutes. A GPC column (Zorbax GF-450) is used to monitor PEG conjugation. At the end of the reaction (as evidenced by the absence of native enzyme), the mixture is diluted with 12 mL of formulation buffer (0.05 M sodium phosphate, 0.85% sodium chloride, pH 7.3) and diafiltered with a Centriprep concentrator (Amicon) to remove the unreacted PEG reactant. Dialfiltration is continued as needed at 4° C. until no more free PEG was detected by mixing equal amount of filtrate and 0.1% PMA (polymethacrylic acid in 0.1 M HCl) to give the product.

### Example 4

### Preparation of Compound (6)

To a solution of C6-thio-LNA-survivin (100 mg, 0.018 mmol) in 60 mL pH 8.0 phosphate buffer is added compound 4 (3.6 g, 0.18 mmol) and the solution was stirred for 1 hour at room temperature. Reaction progress is checked by anion-exchange HPLC. The reaction mixture is filtered through 0.2 micron filter and loaded on Poros anion-exchange column. Product is eluted with a gradient using buffer system 20 mM Tris. HCl 2M NaCl at pH 7.0.

### Example 5

### Preparation of Compound (7)

Compound 4 (8 mg, 0.0014 mmol, oligo eq) is mixed with HS-RGD2 (111 mg, 0.0496 mmol) in 3 mL of buffer (5M urea, 100 mM KH₂PO₄) under nitrogen. The reaction is run for 2 hours. The crude product is purified on Source 15S resin. Column is equilibrated with buffer A (5M urea, 100 mM KH₂PO₄, 25% CH₃CN, pH 6.5). The product is eluted with buffer B (2M KBr). The collected product is desalted on HiPrep desalting column, lyophilized.

### Example 6

### Preparation of Compound (9a)

^{20K}8arm-PEG-SC (compound 8a, 7.30 g, 0.35 mmol) and DIEA (3 mL, 16.8 mmol) were added to a solution of compound 2 (1.82 g, 5.55 mmol) in DMF (25 mL) and DCM (45 mL). The resulting suspension was stirred at room temperature for 5 hours. The reaction mixture was evaporated in vacuo and then precipitated with DCM-ethyl ether (4:1, v/v) at 0° C. The solid was filtered and dissolved in 80 mL of DCM. Alter addition of 20 mL of 0.1 N HCl, the mixture was stirred for 5 minutes, then transferred to a separatory funnel and the organic layer was separated and washed again with 0.1 N HCl (20 mL) and brine (20 mL). The organic layer was dried over anhydrous MgSO₄, filtered and concentrated in vacuo. The residue was precipitated with DCM-Et₂O at 0° C. The solid was filtered and dried in the vacuum oven at 30° C. to give the product: ¹³C NMR d 170.90, 156.66, 155.68, 153.86, 142.41, 133.85, 121.24, 72.96-69.30, 64.08, 53.01, 41.82.

### Example 7

### Preparation of Compound (11a)

To a solution of LNA-Survivin (compound 10, 1.7 μmol) in PBS buffer (5 mL, pH 7.8) is added compound 9a (Mw 20 kDa, 17 μmol) and stirred at room temperature for 5 hours. The reaction mixture is diluted to 50 mL with water and loaded on a Poros HQ, strong anion exchange column (10 mm×1.5 mm, bed volume ˜16 mL) which is pre-equilibrated with 20 mM Tris-HCl buffer, pH 7.4 (buffer A). The column is washed with 3-4 column volumes of buffer A to remove the excess PEG linker. Then the product is eluted with a gradient of 0 to 100% 1 M NaCl in 20 mM Tris-HCl buffer, pH 7.4, buffer B in 10 min, followed by 100% buffer B for 10 rain at a flow rate of 10 mL/min. The eluted product is desalted using HiPrep desalting column (50 mL) and lyophilized to give the product.

### Example 8

### Preparation of Compound (13a)

Compound 11a (0.084 mmol) is added to RGD-K-NH₂ (compound 12, 0.00027 mmol) in 3 mL of sodium phosphate buffer (0.1 M, pH 7.8) with gentle stirring. The solution is stirred at 30° C. for 30 minutes. A GPC column (Zorbax GF-450) is used to monitor PEG conjugation. At the end of the reaction (as evidenced by the absence of native enzyme), the mixture is diluted with 12 mL of formulation buffer (0.05 M sodium phosphate, 0.85% sodium chloride, pH 7.3) and diafiltered with a Centriprep concentrator (Amicon) to remove the unreacted PEG reactant. Dialfiltration is continued as needed at 4° C. until no more free PEG was detected by mixing equal amount of filtrate and 0.1% PMA (polymethacrylic acid in 0.1 M HCl) to give the product.

### Example 9

### Preparation of Compound (14a)

^{20K}8arm-PEG-SC (compound 8a, 7.30 g, 0.35 mmol) and compound 2 (1.82 g, 5.55 mmol) were subjected to the same reaction conditions described in Example 6 to give the product: ¹³C NMR d 170.90, 156.66, 155.68, 153.86, 142.41, 133.85, 121.24, 72.96-69.30, 64.08, 53.01, 41.82.

### Example 10

### Preparation of Compound (14b)

^{20K}4arm-PEG-SC (compound 8b, 6.0 g, 0.29 mmol) and compound 2 (765 mg, 2.33 mmol) were subjected to the same reaction conditions described in Example 6 to give the product: ¹³C NMR d 170.76, 156.53, 155.57, 153.85, 142.37, 133.79, 121.23, 72.44-69.30, 63.99, 52.95, 45.36, 41.82.

### Example 11

### Preparation of Compound (15a)

To a solution of C6-thio-LNA-survivin (compound 10, 120 mg, 0.021 mmol) in 60 mL pH 6.5 phosphate buffer was added compound 14a (2.3 mg, 0.107 mmol) and the solution was stirred for 1 h at room temperature. Reaction progress was checked by anion-exchange HPLC. The reaction mixture was filtered through 0.2 micron filter and loaded on Poros anion-exchange column. Product was eluted with a gradient using buffer system 20 mM Tris. HCl 2M NaCl at pH 7.0. Yield after desalting was 80 mg of oligo eq. calculated from UV.

### Example 12

### Preparation of Compound (17a)

Compound 15a (80 mg oligo eq, 0.0142 mmol) was dissolved in 20 ml of buffer (5M urea, 100 mM KH₂PO₄). The solution was cooled at 0° C. under nitrogen and then the peptide C-TAT (329 mg, 0.198 mmol) was added. The rich yellow color was observed. Continued to stir the reaction for 1.5 hours under nitrogen at 0° C. and then purified by cation-exchange chromatography using the Source 15S resin. Column (10 mm×10 mm) was equilibrated with buffer A (5M urea, 100 mM KH₂PO₄, 25% CH₃CN, pH 6.5) for three column volumes and then the sample was loaded onto the column. The product was eluted with buffer B (2M KBr). The collected product was lyophilized and desalted on HiPrep desalting column with 50 mM pH 7.4 PBS buffer. The desalted solution was then concentrated to about 1 mg/ml (oligo eq) solution. Product yield 21.75 mg.

### Example 13

### Preparation of Compound (19a)

To a solution of 8arm^{20K}-SCPEG (compound 8a, 1 eq) in DMF is added compound 18 (16 eq). Then, DIEA is added (32 eq) and the resulting suspension is stirred at room temperature for 5 h. The reaction mixture is precipitated with DCM/Et₂O at 0° C. The solid is filtered and then is dissolved in water. The crude solid is purified using a C18 reverse-phase chromatography. Product peak is collected and lyophilized to solid.

### Example 14

### Preparation of Compound (20a)

Compound 19a is added to a solution of 2% hydrazine in DMF and the solution is stirred for 4 hours at room temperature. The reaction mixture is loaded on reverse-phase column and purified. The product peak is collected and lyophilized.

### Example 15

### Preparation of Compound (21a)

Compound 20a (1 eq) is dissolved in 20 ml of buffer (5M urea, 100 mM KH₂PO₄). The solution is cooled at 0° C. under nitrogen and then the Oligo-SH (8 eq) is added. Continued to stir the reaction for 1.5 hours under nitrogen at 0° C. and then purified by cation-exchange chromatography using the Source 15S resin. Column (10 mm×10 mm) is equilibrated with buffer A (5M urea, 100 mM KH₂PO₄, 25% CH₃CN, pH 6.5) for three column volumes and then the sample is loaded onto the column. The product is eluted with buffer B (2M KBr). The collected product is lyophilized and desalted on HiPrep desalting column with 50 mM pH 7.4 PBS buffer. The desalted solution is then concentrated to about 1 mg/ml (oligo eq) solution.

1. A compound of the Formula (I): 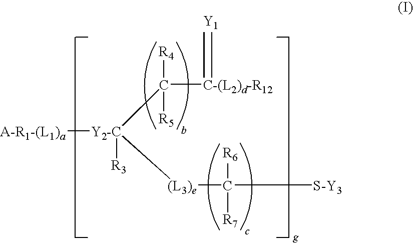
wherein:
R₁ is a substantially non-antigenic water-soluble polymer;
A is a capping group or 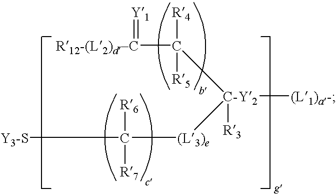
Y₁ and Y′₁ are independently S, O, or NR₂;
Y₂ and Y′₂ are independently S, O, SO, SO₂, NR₂₀;
Y₃ and Y′₃ are independently 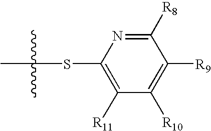
L₁₋₃ and L′₁₋₃ are independently selected bifunctional linkers;
R′₂₋₁₁, R′₂₋₇ and R₂₀ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;
R₁₂ and R′₁₂ are independently selected from a group consisting of hydrogen, hydroxyl, leaving group, functional group, medicinal agent, targeting agent, diagnostic agent, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted arylcarbonyloxy, maleimidyl, vinyl, substituted sulfone, amino, carboxy, mercapto, hydrazide and carbazate;
(a), (a′), (d) and (d′) are independently zero or a positive integer;
(b) and (b′) are independently zero or a positive integer;
(c) and (c′) are independently zero or a positive integer;
(e) and (e′) are independently zero or 1; and
(g) and (g′) are independently zero or 1;
provided that (a) and (g) are not simultaneously zero.

2. The compound of claim 1, wherein R₈₋₁₁ and R′₈₋₁₁ are independently selected from the group consisting of hydrogen, substituted amido, acyl, azido, carboxy, alkyloxycarbonyl, cyano, and nitro.

3. The compound of claim 1, wherein R₁₂ and R′₁₂ are independently selected from the group consisting of H, NH₂, OH, CO₂H, C₁₋₆ alkoxy, C₁₋₆ alkyl, maleimidyl, vinyl, residues of sulfone, mercapto, hydrazide and carbazate.

4. The compound of claim 1, wherein the leaving group is selected from the group consisting of OH, halogens, activated esters, cyclic imide thione, N-hydroxysuccinimidyl, para-nitrophenoxy, N-hydroxyphtalimide, N-hydroxybenzotriazolyl, imidazole, tosyl, mesyl, tresyl, nosyl, C₁₋₆ alkyloxy, C₁₋₆ alkanoyloxy, arylcarbonyloxy, ortho-nitrophenoxy, para-nitrophenoxy, pentafluorophenoxy, 1,3,5-trichlorophenoxy and 1,3,5-trifluorophenoxy.

5. The compound of claim 1, wherein L₁₋₃ and L′₁₋₃ are independently selected from the group consisting of:
—[C(═O)]ᵥ(CR₂₂R₂₃)ₜ[C(═O)]_{v′}—,
—[C(═O)]ᵥ(CR₂₂R₂₃)ₜ—O[C(═O)]_{v′}—,
—[C(═O)]ᵥ(CR₂₂R₂₃)ₜ—NR₂₆[C(═O)]_{v′}—,
—[C(═O)]ᵥO(CR₂₂R₂₃)ₜ[C(═O)]_{v′}—,
—[C(═O)]ᵥO(CR₂₂R₂₃)ₜO[C(═O)]_{v′}—,
—[C(═O)]ᵥO(CR₂₂R₂₃)ₜNR₂₆[C(═O)]_{v′}—,
—[C(═O)]ᵥNR₂₁(CR₂₂R₂₃)ₜ[C(═O)]_{v′}—,
—[C(═O)]ᵥNR₂₁(CR₂₂R₂₃)ₜO[C(═O)]_{v′}—,
—[C(═O)]ᵥNR₂₁(CR₂₂R₂₃)ₜNR₂₆[C(═O)]_{v′}—,
—[C(═O)]ᵥ(CR₂₂R₂₃)ₜO—(CR₂₈R₂₉)_{t′}[C(═O)]_{v′}—,
—[C(═O)]ᵥ(CR₂₂R₂₃)ₜNR₂₆—(CR₂₈R₂₉)_{t′}[C(═O)]_{v′}—,
—[C(═O)]ᵥ(CR₂₂R₂₃)ₜS—(CR₂₈R₂₉)_{t′}[C(═O)]_{v′}—,
—[C(═O)]ᵥO(CR₂₂R₂₃)ₜO—(CR₂₈R₂₉)_{t′}[C(═O)]_{v′}—,
—[C(═O)]ᵥO(CR₂₂R₂₃)ₜNR₂₆—(CR₂₈R₂₉)_{t′}[C(═O)]_{v′}—,
—[C(═O)]ᵥO(CR₂₂R₂₃)ₜS—(CR₂₈R₂₉)_{t′}[C(═O)]_{v′}—,
—[C(═O)]ᵥNR₂₁(CR₂₂R₂₃)ₜO—(CR₂₈R₂₉)_{t′}[C(═O)]_{v′}—,
—[C(═O)]ᵥNR₂₁(CR₂₂R₂₃)ₜNR₂₆—(CR₂₈R₂₉)_{t′}[C(═O)]_{v′}—,
—[C(═O)]ᵥNR₂₁(CR₂₂R₂₃)ₜS—(CR₂₈R₂₉)_{t′}[C(═O)]_{v′}—,
—[C(═O)]ᵥ(CR₂₂R₂₃CR₂₈R₂₉O)ₜNR₂₆[C(═O)]_{v′}—,
—[C(═O)]ᵥ(CR₂₂R₂₃CR₂₈R₂₉O)ₜ[C(═O)]_{v′}—,
—[C(═O)]ᵥO(CR₂₂R₂₃CR₂₈R₂₉O)ₜNR₂₆[C(═O)]_{v′}—,
—[C(═O)]ᵥO(CR₂₂R₂₃CR₂₈R₂₉O)ₜ[C(═O)]_{v′}—,
—[C(═O)]ᵥNR₂₁(CR₂₂R₂₃CR₂₈R₂₉O)ₜNR₂₆[C(═O)]_{v′}—,
—[C(═O)]ᵥNR₂₁(CR₂₂R₂₃CR₂₈R₂₉O)ₜ[C(═O)]_{v′}—,
—[C(═O)]ᵥ(CR₂₂R₂₃CR₂₈R₂₉O)ₜ(CR₂₄R₂₅)_{t′}[C(═O)]_{v′}—,
—[C(═O)]ᵥO(CR₂₂R₂₃CR₂₈R₂₉O)ₜ(CR₂₄R₂₅)_{t′}[C(═O)]_{v′}—,
—[C(═O)]ᵥNR₂₁(CR₂₂R₂₃CR₂₈R₂₉O)ₜ(CR₂₄R₂₅)_{t′}[C(═O)]_{v′}—,
—[C(═O)]ᵥ(CR₂₂R₂₃CR₂₈R₂₉O)ₜ(CR₂₄R₂₅)_{t′}O[C(═O)]_{v′}—,
—[C(═O)]ᵥ(CR₂₂R₂₃)ₜ(CR₂₄R₂₅CR₂₈R₂₉O)_{t′}[C(═O)]_{v′}—,
—[C(═O)]ᵥ(CR₂₂R₂₃)ₜ(CR₂₄R₂₅CR₂₈R₂₉O)_{t′}NR₂₆[C(═O)]_{v′}—,
—[C(═O)]ᵥO(CR₂₂R₂₃CR₂₈R₂₉O)ₜ(CR₂₄R₂₅)_{t′}O[C(═O)]_{v′}—,
—[C(═O)]ᵥO(CR₂₂R₂₃)ₜ(CR₂₄R₂₅CR₂₈R₂₉O)_{t′}[C(═O)]_{v′}—,
—[C(═O)]ᵥO(CR₂₂R₂₃)ₜ(CR₂₄R₂₅CR₂₈R₂₉O)_{t′}NR₂₆[C(═O)]_{v′}—,
—[C(═O)]ᵥNR₂₁(CR₂₂R₂₃CR₂₈R₂₉O)ₜ(CR₂₄R₂₅)_{t′}O[C(═O)]_{v′}—,
—[C(═O)]ᵥNR₂₁(CR₂₂R₂₃)ₜ(CR₂₄R₂₅CR₂₈R₂₉O)_{t′}[C(═O)]_{v′}—,
—[C(═O)]ᵥNR₂₁(CR₂₂R₂₃)ₜ(CR₂₄R₂₅CR₂₈R₂₉O)_{t′}NR₂₆[C(═O)]_{v′}—, 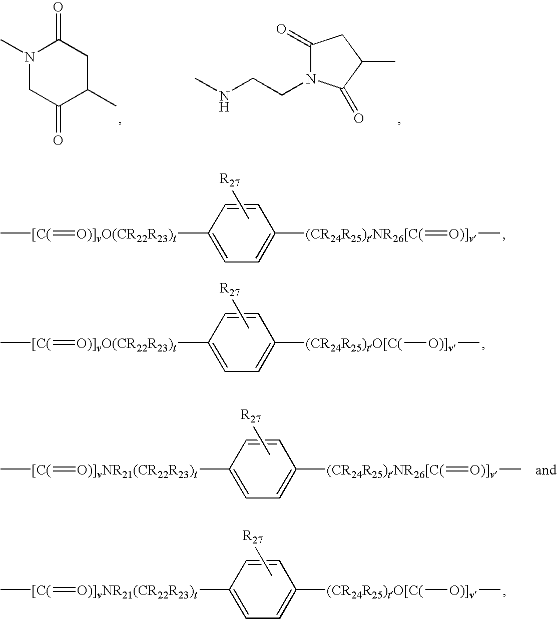
wherein:
R₂₁₋₂₉ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
(t) and (t′) are independently zero or a positive integer; and
(v) and (v′) are independently zero or 1.

6. The compound of claim 1, wherein L₁₋₃ and L′₁₋₃ are independently selected from the group consisting of:
—[C(═O)]ᵣNH(CH₂)₂CH═N—NHC(═O)—(CH₂)₂—,
—[C(═O)]ᵣNH(CH₂)₂(CH₂CH₂O)₂(CH₂)₂NH[C(═O)]_{r′}—,
—[C(═O)]ᵣNH(CH₂CH₂)(CH₂CH₂O)₂NH[C(═O)]_{r′}—,
—[C(═O)]ᵣNH(CH₂CH₂)ₛNH(CH₂CH₂)_{s′}[C(═O)]_{r′}—,
—[C(═O)]ᵣNH(CH₂CH₂)ₛS(CH₂CH₂)_{s′}[C(═O)]_{r′}—,
—[C(═O)]ᵣNH(CH₂CH₂)(CH₂CH₂O)[C(═O)]_{r′}—,
—[C(═O)]ᵣNH(CH₂CH₂)ₛO(CH₂CH₂)_{s′}[C(═O)]_{r′}—,
—[C(═O)]ᵣNH(CH₂CH₂O)(CH₂CH₂)NH[C(═O)]_{r′}—,
—[C(═O)]ᵣNH(CH₂CH₂O)₂(CH₂)[C(═O)]_{r′}—,
—[C(═O)]ᵣNH(CH₂CH₂O)ₛ(CH₂)_{s′}[C(═O)]_{r′}—,
—[C(═O)]ᵣNHCH₂CH₂NH[C(═O)]_{r′}—,
—[C(═O)]ᵣNH(CH₂CH₂)₂O[C(═O)]_{r′}—,
—[C(═O)]ᵣNH(CH₂CH₂O)[C(═O)]_{r′}—,
—[C(═O)]ᵣNH(CH₂CH₂O)₂[C(═O)]_{r′}—,
—[C(═O)]ᵣNH(CH₂)₃[C(═O)]_{r′}—,
—[C(═O)]ᵣO(CH₂CH₂O)₂(CH₂)[C(═O)]_{r′}—,
—[C(═O)]ᵣO(CH₂)₂NH(CH₂)₂[C(═O)]_{r′}—,
—[C(═O)]ᵣO(CH₂CH₂O)₂NH[C(═O)]_{r′}—,
—[C(═O)]ᵣO(CH₂)₂O(CH₂)₂[C(═O)]_{r′}—,
—[C(═O)]ᵣO(CH₂)₂S(CH₂)₂[C(═O)]_{r′}—,
—[C(═O)]ᵣO(CH₂CH₂)NH[C(═O)]_{r′}—,
—[C(═O)]ᵣO(CH₂CH₂)O[C(═O)]_{r′}—,
—[C(═O)]ᵣO(CH₂)₃NH[C(═O)]_{r′}—,
—[C(═O)]ᵣO(CH₂)₃O[C(═O)]_{r′}—,
—[C(═O)]ᵣO(CH₂)₃[C(═O)]_{r′}—,
—[C(═O)]ᵣCH₂NHCH₂[C(═O)]_{r′}—,
—[C(═O)]ᵣCH₂OCH₂[C(═O)]_{r′}—,
—[C(═O)]ᵣCH₂SCH₂[C(═O)]_{r′}—,
—[C(═O)]ᵣS(CH₂)₃[C(═O)]_{r′}—,
—[C(═O)]ᵣ(CH₂)₃[C(═O)]_{r′}—, 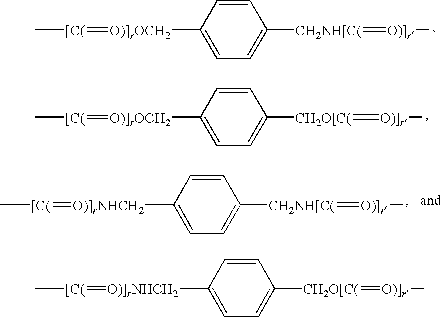
wherein (r) and (r′) are independently zero or 1, provided that both are not zero simultaneously.

7. The compound of claim 1, wherein L₁₋₃ and L′₁₋₃ are independently selected from the group consisting of amino acids, amino acid derivatives, and peptides.

8. The compound of claim 1, wherein L₁₋₃ and L′₁₋₃ are independently selected from the group consisting of: 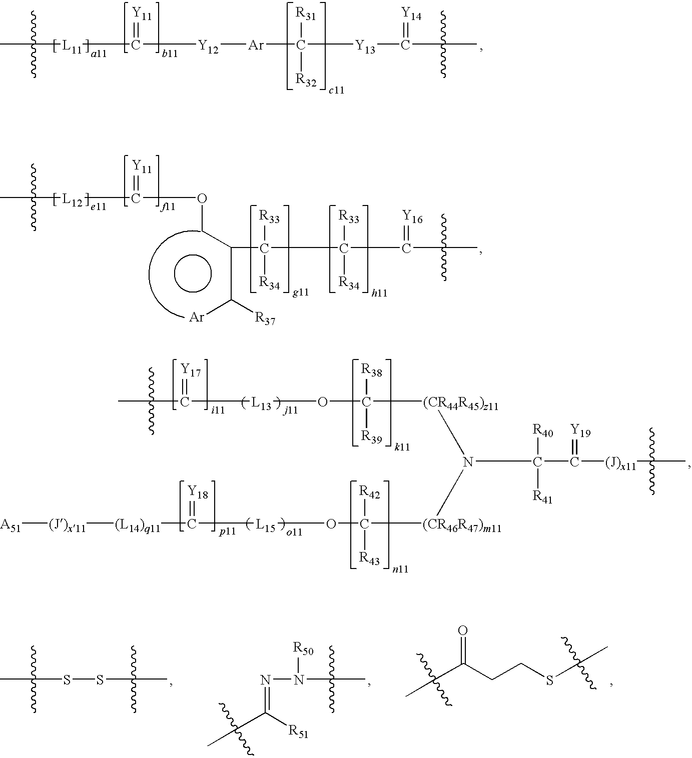
-Val-Cit-,
-Gly-Phe-Leu-Gly-,
-Ala-Leu-Ala-Leu-,
-Phe-Lys-, 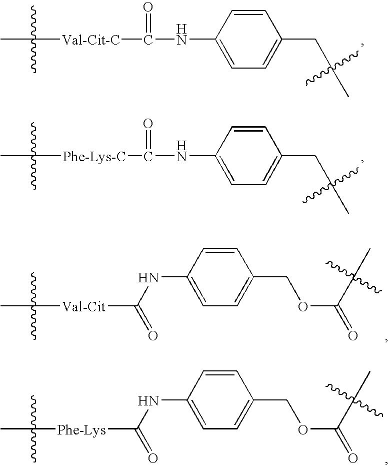
-Val-Cit-C(═O)—CH₂OCH₂—C(═O)—,
-Val-Cit-C(═O)—CH₂SCH₂—C(═O)—, and
—NHCH(CH₃)—C(═O)—NH(CH₂)₆—C(CH₃)₂—C(═O)—
wherein,
Y₁₁₋₁₉ are independently O, S or NR₄₈;
R₃₁₋₄₈, R₅₀₋₅₁ and A₅₁ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cyloalkyls, aryls, substituted aryls, aralkyls, C₁₋₆ heteroalkyls, substituted C₁₋₆ heteroalkyls, C₁₋₆ alkoxy, phenoxy and C₁₋₆ heteroalkoxy;
Ar is an aryl or heteroaryl moiety;
L₁₁₋₁₅ are independently selected bifunctional spacers;
J and J′ are independently selected from the group consisting of moieties actively transported into a target cell, hydrophobic moieties, bifunctional linking moieties and combinations thereof;
(c11), (h11), (k11), (z11), (m11) and (n11) are independently selected positive integers;
(a11), (e11), (g11), (j11), (o11) and (q11) are independently zero or a positive integer; and
(b11), (x11), (x′11), (f11), (i11) and (p11) are independently zero or one.

9. The compound of claim 1, wherein A is selected from the group consisting of H, NH₂, OH, CO₂H, C₁₋₆ alkoxy and C₁₋₆ alkyl.

10. The compound of claim 1 having the formula:

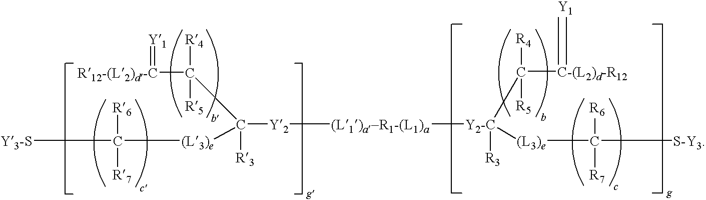

11. The compound of claim 1 having the formula (II) 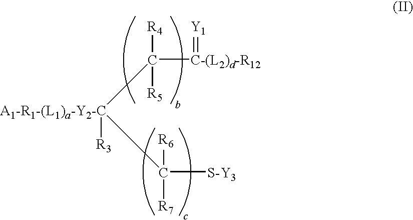
wherein
A₁ is a capping group or 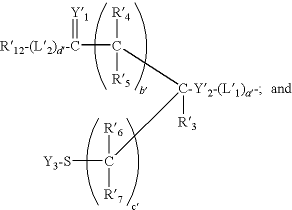
all other variables are the same as defined in claim 1.

12. The compound of claim 1 wherein L₁ and L′₁ are lysine.

13. The compound of claim 1, wherein R₁ comprises a linear, terminally branched or multi-armed polyalkylene oxide.

14. The compound of claim 13, wherein the polyalkylene oxide is selected from the group consisting of polyethylene glycol and polypropylene glycol.

15. The compound of claim 13, wherein the polyalkylene oxide is selected from the group consisting of:
—Y₇₁—(CH₂CH₂O)ₙ—CH₂CH₂—Y₇₁—,
—Y₇₁—(CH₂CH₂O)ₙ—CH₂C(═Y₇₂)—Y₇₁—,
—Y₇₁—C(═Y₇₂)—(CH₂)ₐ₇₁—Y₇₃—(CH₂CH₂O)ₙ—CH₂CH₂—Y₇₃—(CH₂)ₐ₇₁—C(═Y₇₂)—Y₇₁—, and
—Y₇₁—(CR₇₁R₇₂)ₐ₇₂—Y₇₃—(CH₂)_{b71}—O—(CH₂CH₂O)ₙ—(CH₂)_{b71}—Y₇₃—(CR₇₁R₇₂)ₐ₇₂—Y₇₁—,
wherein:
Y₇₁ and Y₇₃ are independently O, S, SO, SO₂, NR₇₃ or a bond;
Y₇₂ is O, S, or NR₇₄;
R₇₁, R₇₁, R₇₃, and R₇₄ are independently selected from the same moieties which can be used for R₂;
(a71), (a72), and (b71) are independently zero or a positive integer; and
(n) is an integer from about 10 to about 2300.

16. The compound of claim 13, wherein the polyalkylene oxide is a polyethylene glycol of the formula, —O—(CH₂CH₂O)ₙ—
wherein (n) is an integer from about 10 to about 2,300.

17. The compound of claim 1, wherein R₁ has an average molecular weight from about 200 to about 250,000 daltons.

18. (canceled)

19. The compound of claim 1, wherein R₁ has an average molecular weight from about 2,000 to about 100,000 daltons.

20. (canceled)

21. The compound of claim 1, wherein R₁ has an average molecular weight from about 5,000 to about 25,000 daltons or from about 20,000 to about 45,000 daltons.

22. A compound of claim 1 selected from the group consisting of: 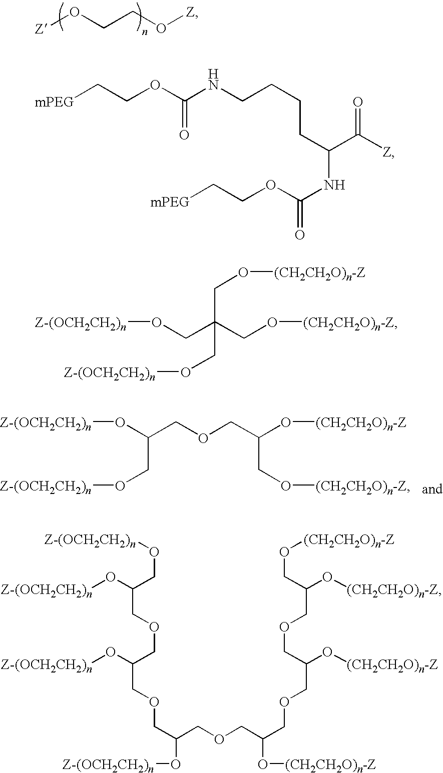
wherein
mPEG is CH₃O—(CH₂CH₂O)ₙ— wherein (n) is an integer from about 10 to about 2,300; and
Z and Z′ are independently capping groups or 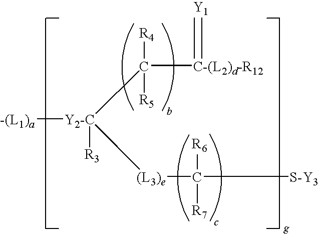
provided that at least one Z is not a capping group.

23. The compound of claim 1 wherein R₂₋₇ and R′₂₋₇ are independently selected from the group consisting of hydrogen, methyl, ethyl and isopropyl.

24. A compound of claim 1 having the formula: 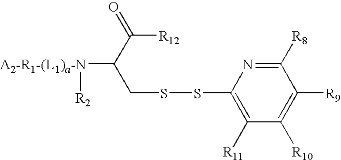
wherein,
A₂ is a capping group or 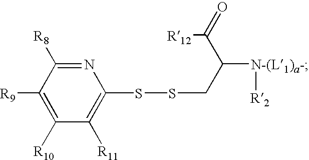
and
all other variables are the same as defined in claim 1.

25. (canceled)

26. A compound of claim 1 selected from the group consisting of: 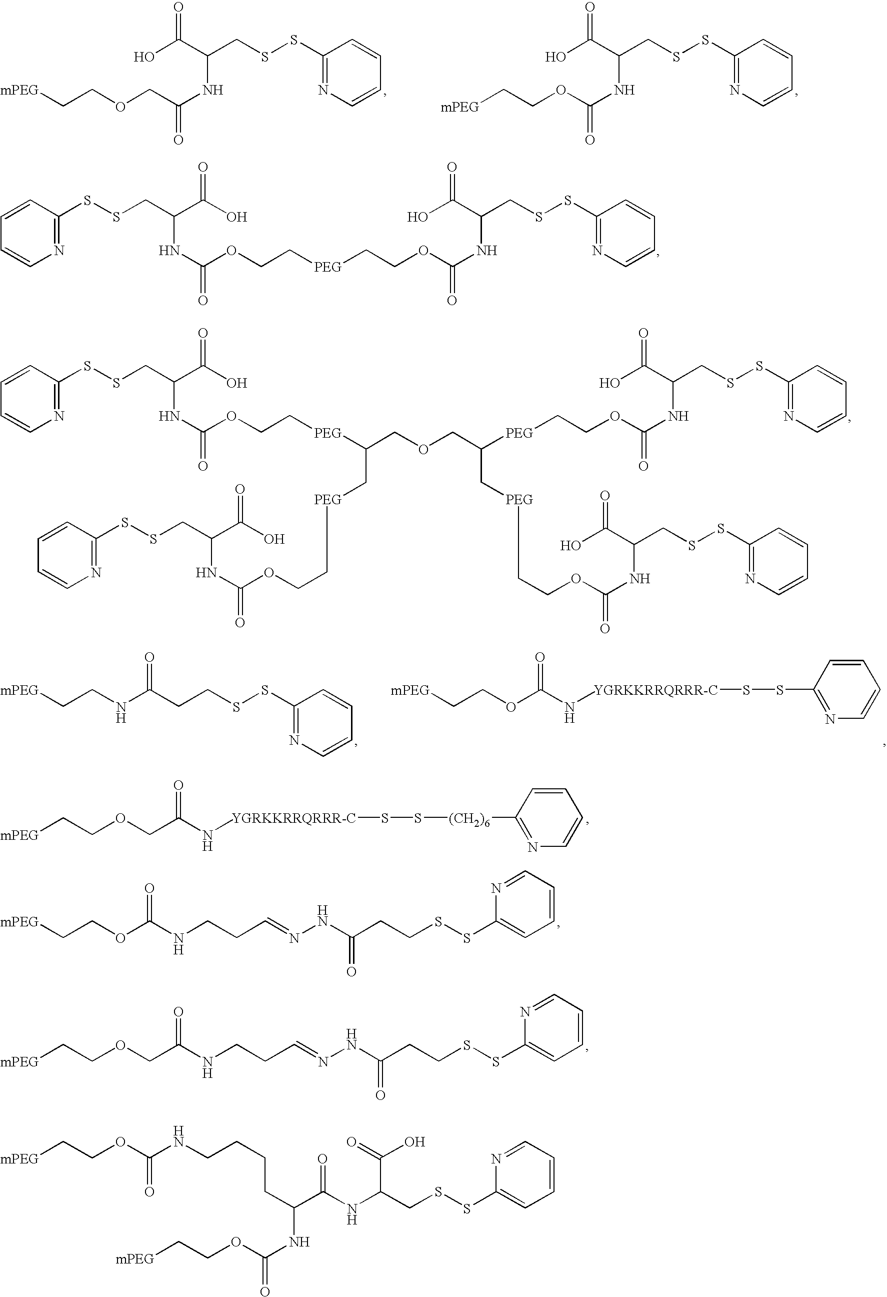 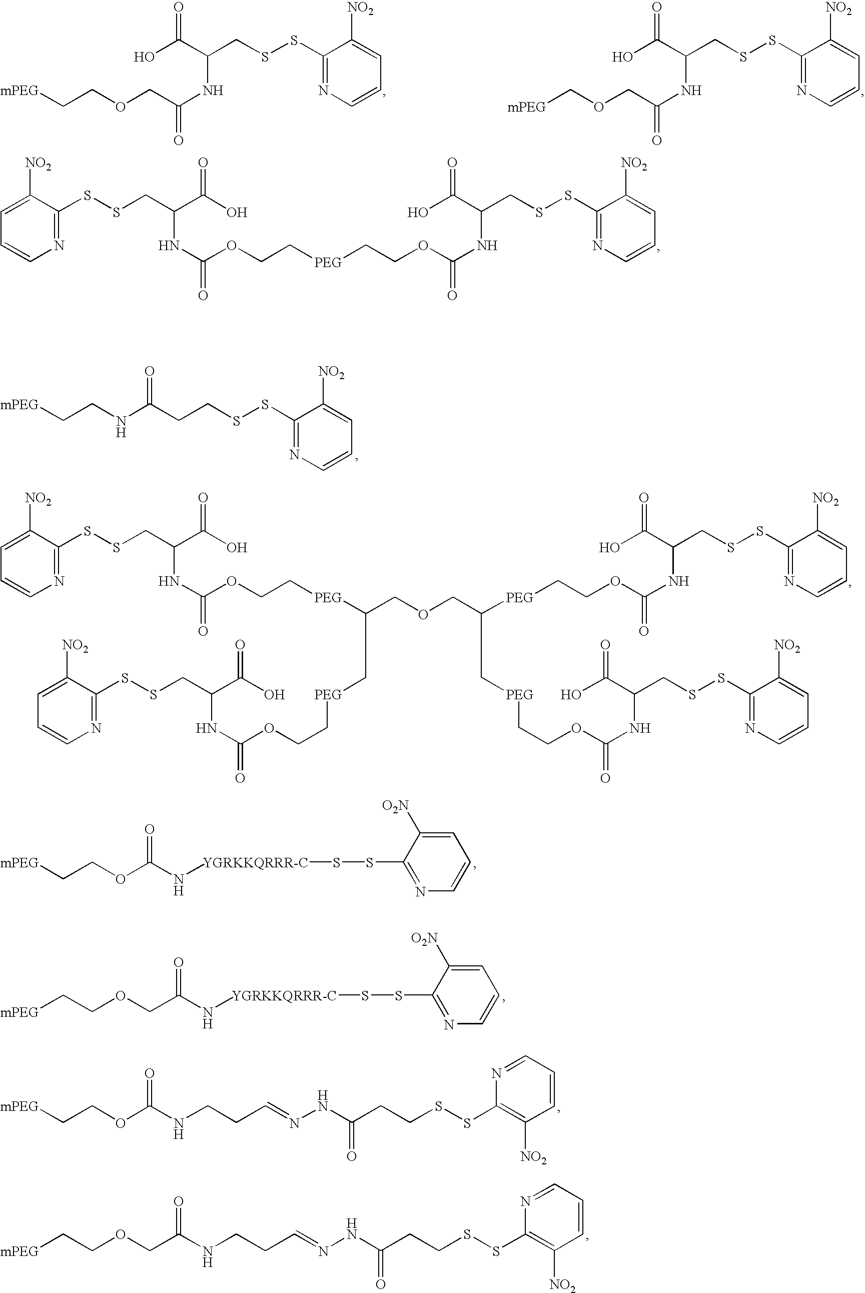 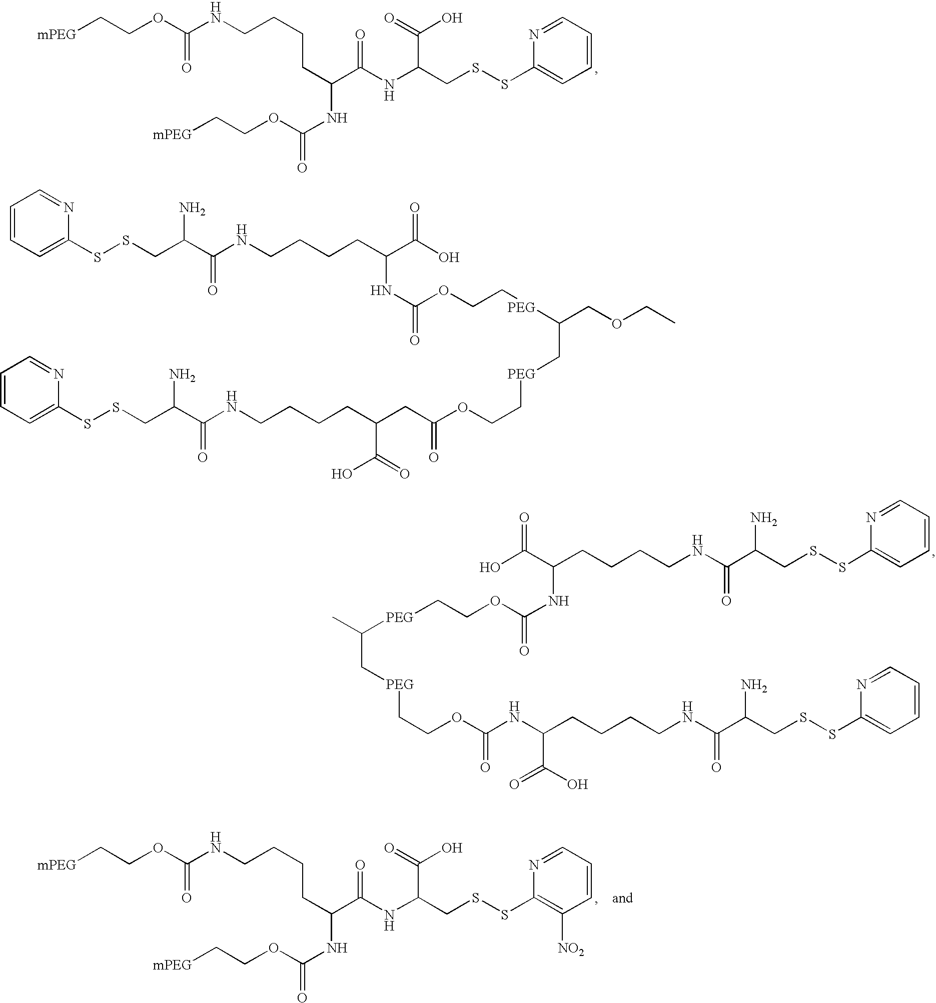 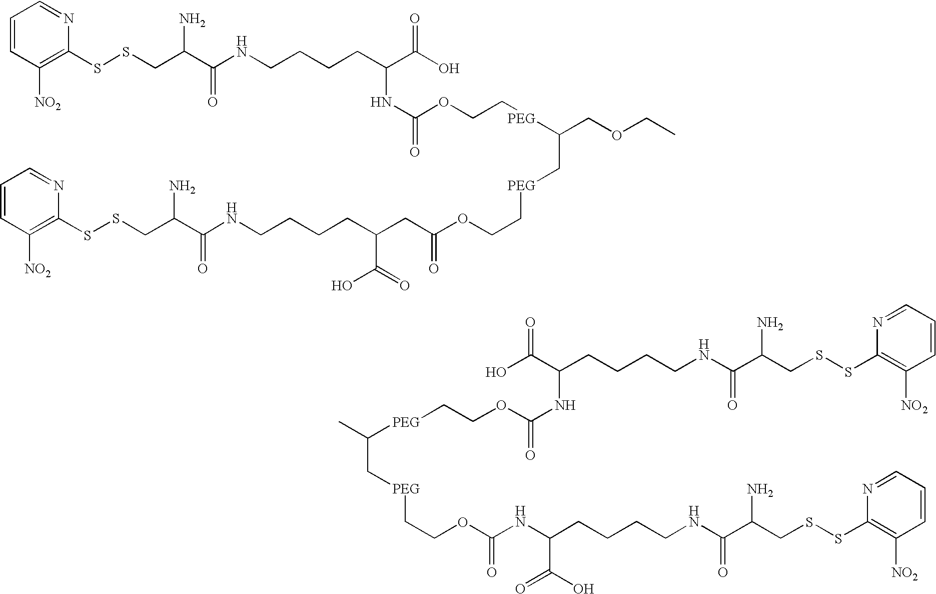
wherein:
mPEG has the formula CH₃O(CH₂CH₂O)ₙ—;
PEG has the formula —O(CH₂CH₂O)ₙ—, and
(n) is an integer from about 10 to about 2,300.

27. A method of preparing a polymeric compound containing a pyridyl disulfide moiety comprising:
reacting a polymeric compound of Formula (III):
A₄-R₁-M₁ (III)
with a compound of Formula (VI): 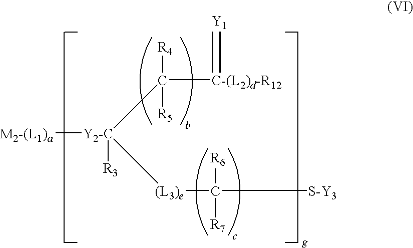
under conditions sufficient to form a compound of the formula (V): 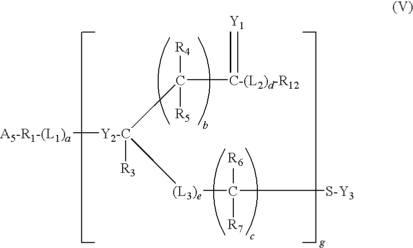
wherein:
R₁ is a substantially non-antigenic water-soluble polymer;
A₄ is a capping group or M₁;
A₅ is a capping group or 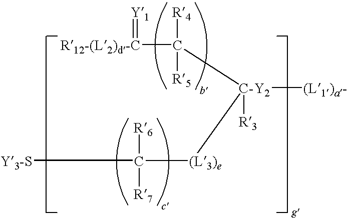
M₁ is OH or a leaving group;
M₂ is —OH, SH, or —NHR₉₀;
Y₁ and Y′₁ are independently S, O, or NR₂;
Y₂ and Y′₂ are independently S, O, SO, SO₂, NR₂₀;
Y₃ and Y′₃ are independently 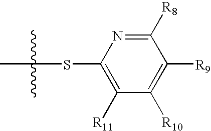
L₁₋₃ and L′₁₋₃ are independently selected bifunctional linkers;
R₂₋₁₁, R′₂₋₁₁, R₂₀ and R₉₀ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;
R₁₂ and R′₁₂ are independently selected from a group consisting of hydrogen, hydroxyl, leaving group, functional group, medicinal agent, targeting agent, diagnostic agent, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted arylcarbonyloxy, maleimidyl, vinyl, substituted sulfone, amino, carboxy, mercapto, hydrazide and carbazate;
(a), (a′), (d) and (d′) are independently zero or a positive integer;
(b) and (b′) are independently zero or a positive integer;
(c) and (c′) are independently zero or a positive integer;
(e) and (e′) are independently zero or 1; and
(g) and (g′) are independently zero or 1;
provided that (a) and (g) are not simultaneously zero.

28. The method of claim 27 further comprising reacting the compound of Formula (V) with a sulfhydryl group-containing moiety under conditions sufficient to form a polymer conjugate.

29. The method of claim 28, wherein the sulfhydryl group-containing moiety is a biologically active moiety selected from the group consisting of pharmaceutically active compounds, enzymes, proteins, oligonucleotides, antibodies, monoclonal antibodies, single chain antibodies and peptides.

30. The polymeric conjugate prepared by the method of claim 28.

31. A method of treating a mammal, comprising administering an effective amount of the compound of claim 30 to a patient in need thereof.

32. A compound having the formula: 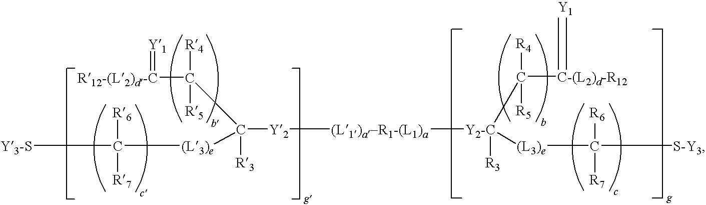
wherein
R₁ is a substantially non-antigenic water-soluble polymer;
Y₁ and Y′ₗ are independently S, O, or NR₂;
Y₂ and Y′₂ are independently S, O, SO, SO₂, NR₂₀;
Y₃ and Y′₃ are independently sulfhydryl group-containing biologically active moieties linked via the sulfhydryl group;
L₁₋₃ and L′₁₋₃ are independently selected bifunctional linkers;
R₂₋₇, R′₂₋₇, and R₂₀ are independently selected from the group consisting of hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy;
R₁₂ and R′₁₂ are independently selected from a group consisting of hydrogen, hydroxyl, leaving group, functional group, medicinal agent, targeting agent, diagnostic agent, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted arylcarbonyloxy, maleimidyl, vinyl, substituted sulfone, amino, carboxy, mercapto, hydrazide and carbazate;
(a), (a′), (d) and (d′) are independently zero or a positive integer;
(b) and (b′) are independently zero or a positive integer;
(c) and (c′) are independently zero or a positive integer;
(e) and (e′) are independently zero or 1; and
(g) and (g′) are zero or 1.

33. A compound of claim 32 selected from the group consisting of: 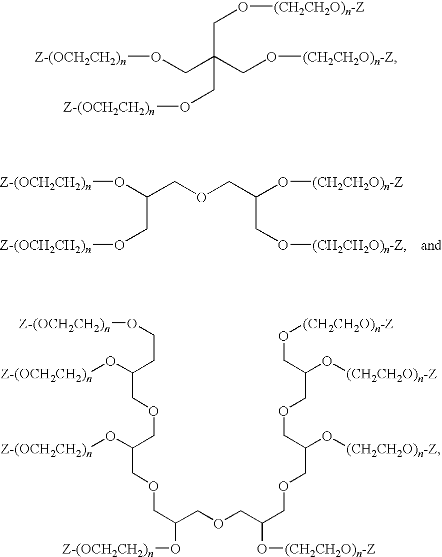
wherein
(n) is an integer from about 10 to about 2,300; and
Z and Z′ are independently [chemical structure]
